(19) United States
(12) Patent Application Publication
(10) Pub. No.: US 2025/0375203 A1
(43) Pub. Date: Dec. 11, 2025

(54) LIGATION DEVICE HAVING STRUCTURE FOR MOVING RETRACTING MEMBER CONFIGURED TO DRAW THREAD INTO DEVICE BODY TO LIGATE LIGATION TARGET WITH THE THREAD

(71) Applicant: Brother Kogyo Kabushiki Kaisha, Nagoya (JP)

(72) Inventors: Naokatsu Osawa, Nagoya (JP); Junji Yamano, Kariya (JP); Ryuta Iijima, Nagoya (JP); Masashi Ichihashi, Mizuho (JP)

(21) Appl. No.: 19/309,160

(22) Filed: Aug. 25, 2025

Related U.S. Application Data
(63) Continuation of application No. PCT/JP2024/009519, filed on Mar. 12, 2024.

(30) Foreign Application Priority Data
Mar. 24, 2023 (JP) 2023-048626

(51) Int. Cl. A61B 17/12; A61B 17/00

(52) U.S. Cl. CPC A61B 17/12013; A61B 2017/00212; A61B 2017/00398

(57) ABSTRACT

A ligation device includes: a body having a cylindrical shape and extending in a longitudinal direction; a holder disposed at one end of the body in the longitudinal direction; a retractor having a part accommodated inside the body; and a drive unit connected to an other end of the body in the longitudinal direction. The holder is configured to hold a ligation target. The part of the retractor is movable toward the other end of the body in the longitudinal direction to draw a thread into an interior of the body for ligating the ligation target held by the holder. The drive unit includes a first motor for moving the retractor in the longitudinal direction relative to the body.

### REFERENCE TO RELATED APPLICATIONS

This is a by-pass continuation application of International Application No. PCT/JP2024/009519 filed on Mar. 12, 2024 which claims priority from Japanese Patent Application No. 2023-048626 filed on Mar. 24, 2023. The entire contents of the International Application and the priority application are incorporated herein by reference.

### BACKGROUND ART

There has been known a ligation device that uses thread to ligate a ligation target within a living body. The ligation device includes a first extendable member, an advancing member, a second extendable member, and a snaring member. Each of the first extendable member and second extendable member has a lumen. The advancing member has a distal end that holds a loop to which a thread is attached. The advancing member is disposed within the lumen of the first extendable member and is movable along the lumen of the first extendable member. The snaring member has a distal end on which a snaring part is provided. The snaring member is disposed within the lumen of the second extendable member and is movable along the lumen of the second extendable member. An operator manually performs operations for moving the first extendable member, the advancing member, the second extendable member, and the snaring member.

A method of using the ligation device to ligate a ligation target with a thread is as follows. The first and second extendable members move toward the ligation target in accordance with operator's operations. The movement of the first and second extendable members is halted after distal ends of the first and second extendable members have moved past the ligation target. Next, the advancing member and the snaring member move in accordance with operator's operations. The loop held on the distal end of the advancing member protrudes from the distal end of the first extendable member. The snaring part disposed on the distal end of the snaring member protrudes from the distal end of the second extendable member. The snaring part snares the loop. Next, the advancing member and the snaring member are moved toward the near side of the ligation target in response to operator's operations. The loop ensnared by the snaring member detaches from the advancing member. The ligation target is now surrounded by the thread. Through this process, the ligation target can be ligated by the thread.

### SUMMARY

With the ligation device, the operator manually performs operations for ligating a ligation target with a thread. Consequently, irregularities in operations could hinder the ligation target from being stably ligated.

In view of the foregoing, it is an object of the present disclosure to provide a ligation device capable of stably ligating a ligation target with a thread.

In order to attain the above and other objects, the present disclosure provides a ligation device including a body, a holder, a retractor, and a drive unit. The body has a cylindrical shape and extends in a longitudinal direction. The body has one end and an other end opposite each other in the longitudinal direction. The holder is disposed at the one end of the body and is configured to hold a ligation target. The retractor has a part accommodated inside the body. The retractor is movable toward the other end of the body in the longitudinal direction to draw a thread into an interior of the body, the thread being to be used for ligating the ligation target held by the holder. The drive unit is connected to the other end of the body. The drive unit includes a first motor for moving the retractor in the longitudinal direction relative to the body.

With the above structure, the ligation device is configured to draw the thread for ligating the ligation target into the body by the retractor, and ligate the ligation target by the thread. By driving the retractor with the first motor, the ligation device can perform a stable operation to pull the thread for ligating the ligation target into the body. Hence, the ligation device can stably ligate the ligation target with the thread.

### BRIEF DESCRIPTION OF DRAWINGS

FIG. 1 is a perspective view of a ligation device 1A.

FIG. 2 is a cross-sectional view taken along a line II-II in FIG. 1 when viewed from a direction of arrows.

FIG. 3 is a cross-sectional view taken along a line III-III in FIG. 1 when viewed from a direction of arrows.

FIG. 4 is a cross-sectional view taken along a line IV-IV in FIG. 1 when viewed from a direction of arrows.

FIG. 5 is a perspective view of a forming part 2B.

FIG. 6 is a perspective view of a retractor 4, a knot pusher 5, and a drive unit 7.

FIG. 7 is a side view of the retractor 4, the knot pusher 5, and the drive unit 7.

FIG. 8 is a rear view of the drive unit 7.

FIG. 9 is a perspective view of a first retracting member 4A and a first drive mechanism 7A.

FIG. 10 is an exploded perspective view of a first hook support part 70A.

FIG. 11 is a cross-sectional view taken along a line XI-XI in FIG. 9 when viewed from a direction of arrows.

FIG. 12 is a perspective view of a second retracting member 4B and a second drive mechanism 7B.

FIG. 13 is an exploded perspective view of a second hook support part 70B.

FIG. 14 is a cross-sectional view taken along a line XIV-XIV in FIG. 12 when viewed from a direction of arrows.

FIG. 15 is a perspective view of the knot pusher 5 and a third drive mechanism 7C.

FIG. 16 is an exploded perspective view of a pusher support part 70C.

FIG. 17 is a perspective view of a feeder 6.

FIG. 18 is a view for describing a first step of a ligation process.

FIG. 19 is a view for describing a second step of the ligation process.

FIG. 20 is a view for describing a third step of the ligation process.

FIG. 21 is a view for describing a fourth step of the ligation process.

FIG. 22 is a view for describing a fifth step of the ligation process.

FIG. 23 is a view for describing a sixth step of the ligation process.

FIG. 24 is a view for describing a seventh step of the ligation process.

FIG. 25 is a view for describing an eighth step of the ligation process.

FIG. 26 is a view for describing a ninth step of the ligation process.

FIG. 27 is a view for describing a tenth step of the ligation process.

FIG. 28 is a view illustrating a state in which a thread T is wound around a ligation target S.

FIG. 29 is a view for describing an eleventh step of the ligation process.

FIG. 30 is another view illustrating a state in which the thread Tis wound around the ligation target S.

FIG. 31 is a view for describing a twelfth step of the ligation process.

FIG. 32 is a view for describing a thirteenth step of the ligation process.

FIG. 33 is a view for describing a fourteenth step of the ligation process.

FIG. 34 is a view for describing a first step of a repositioning process.

FIG. 35 is a view for describing a second step of the repositioning process.

FIG. 36 is a view for describing a third step of the repositioning process.

FIG. 37 is a view for describing a fourth step of the repositioning process.

FIG. 38 is a view for describing a fifth step of the repositioning process.

FIG. 39 is a view for describing a sixth step of the repositioning process.

FIG. 40 is a view for describing a seventh step of the repositioning process.

FIG. 41 is a perspective view of the first retracting member 4A and a first drive mechanism 8A.

FIG. 42 is a plan view of the first retracting member 4A and the first drive mechanism 8A.

FIG. 43 is an exploded perspective view of a first hook support part 80.

FIG. 44 is a perspective view of the knot pusher 5 and a third drive mechanism 8C.

FIG. 45 is a plan view of the knot pusher 5 and the third drive mechanism 8C.

FIG. 46 is an exploded perspective view of a pusher support part 85.

FIG. 47 is a side view of a second rotary body 86.

FIGS. 48A through 48C are views for describing operations of the first drive mechanism 8A.

FIGS. 49A through 49C are views for describing operations of the first drive mechanism 8A after the operations in FIG. 48C.

FIGS. 50A through 50C are views for describing operations of the third drive mechanism 8C.

FIGS. 51A through 51C are views for describing operations of the third drive mechanism 8C after the operations in FIG. 50C.

### DESCRIPTION

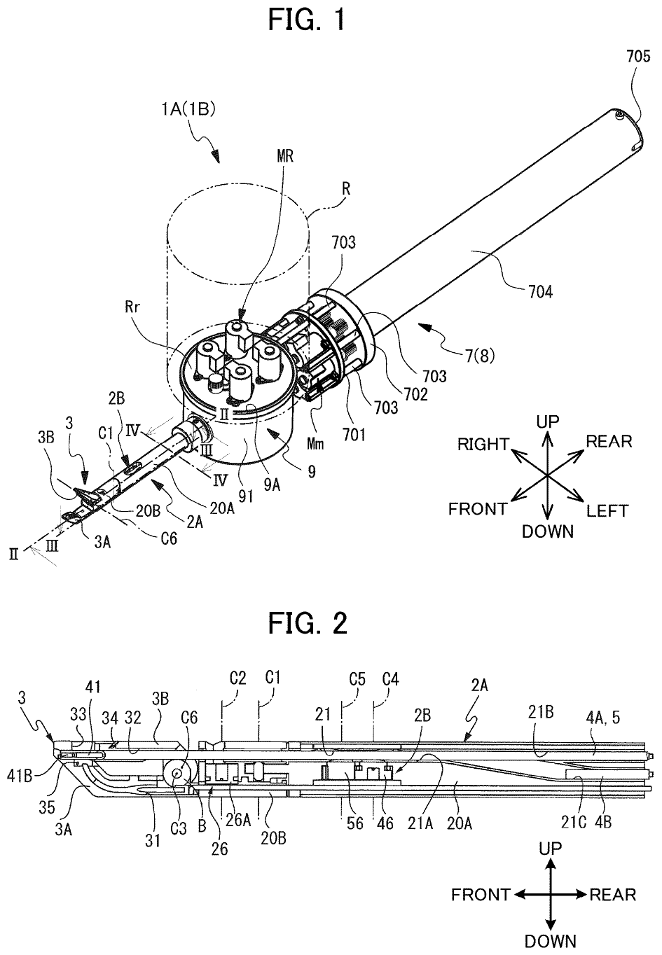

Hereinafter, ligation devices according to embodiments of the present disclosure will be described with reference to the accompanying drawings. The referenced drawings are used to describe technical features that could be employed in in the present disclosure. Configurations of the devices described herein are merely illustrative examples and are not intended to be limited to those examples. The upper, lower, lower-left, upper-right, lower-right, and upper-left sides in FIG. 1 are the upper, lower, front, rear, left, and right sides of each ligation device, respectively.

### <Overview of the Ligation Device>

The ligation device according to each embodiment of the present disclosure is a device for ligating a ligation target S with a thread T. As an example, the ligation target S is a part of a living body, such as a blood vessel. The ligation device is connected to and used with a surgical support robot R (see FIG. 1) for performing surgeries using minimally invasive techniques.

### First Embodiment (Ligation Device 1A)

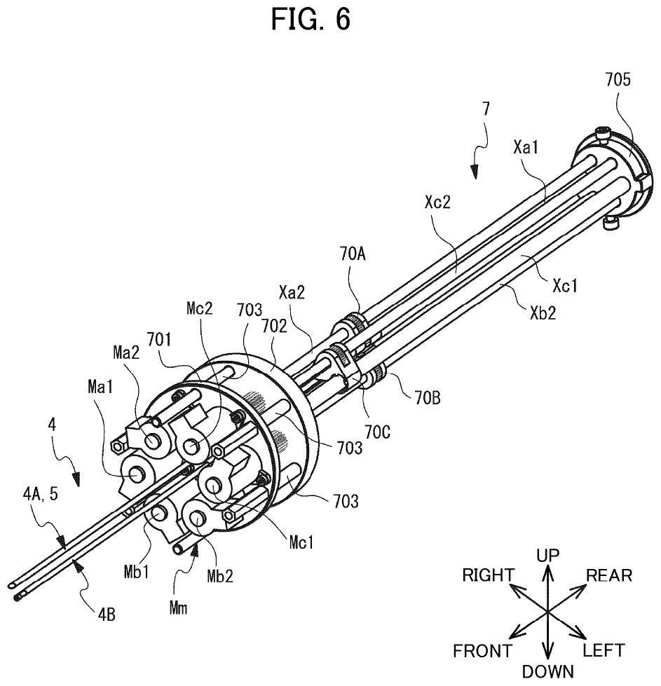

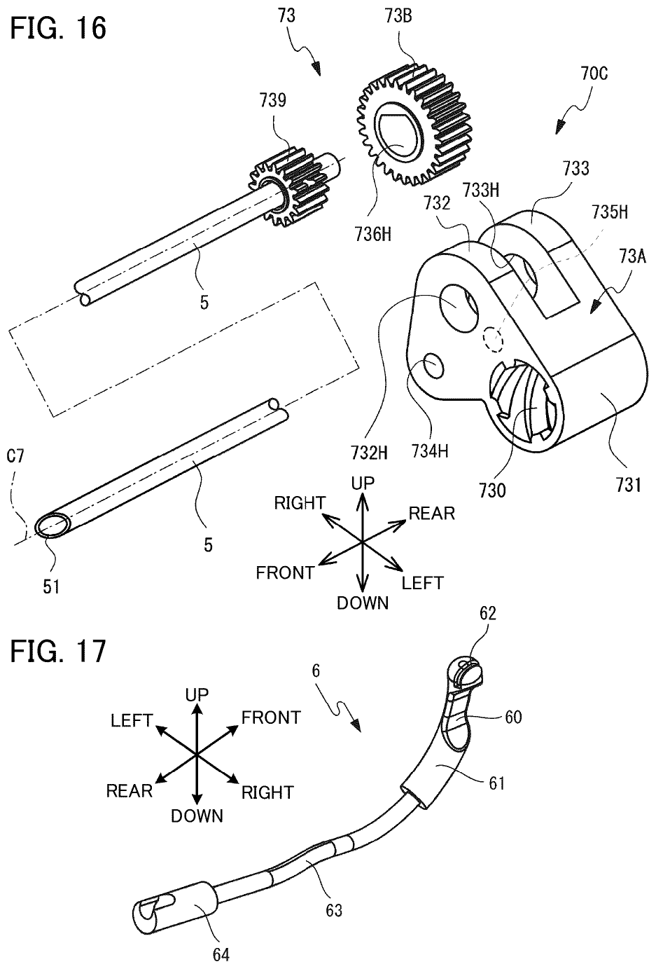

FIG. 1 illustrates a ligation device 1A according to a first embodiment as one of the embodiments of the present disclosure. The ligation device 1A includes a body 2A, a forming part 2B, a holder 3, a retractor 4 (see FIG. 6), a knot pusher 5 (see FIG. 6), a feeder 6 (see FIG. 17), a drive unit 7, and a robot connector 9.

### <Body 2A>

The body 2A has a cylindrical shape that extends in a front-rear direction. The front-rear direction corresponds to a longitudinal direction of the body 2A. The body 2A includes a base 20A and a pivoting part 20B. The pivoting part 20B is supported on a front end of the base 20A. The pivoting part 20B is pivotable about a first axis C1 that extends in an up-down direction. Hereinafter, description will be made based on an assumption that the base 20A and the pivoting part 20B are arranged in line in the front-rear direction. As illustrated in FIGS. 2, 3, and 4, a plurality of insertion holes is formed inside the body 2A. Specifically, a first insertion hole 21, a second insertion hole 22A, a third insertion hole 22B, a fourth insertion hole 23A, a fifth insertion hole 23B, a sixth insertion hole 24A, a seventh insertion hole 24B, and an eighth insertion hole 25 are formed inside the body 2A.

The first insertion hole 21 is arranged in a center of the body 2A in a left-right direction. As illustrated in FIG. 2, the first insertion hole 21 has an extended portion 21A, a first branching portion 21B, and a second branching portion 21C. The extended portion 21A extends rearward from a front end of the body 2A. The first branching portion 21B extends rearward from a rear end of the extended portion 21A. The second branching portion 21C extends diagonally downward and rearward from the rear end of the extended portion 21A and then bends and extends rearward. The first insertion hole 21 branches into the first branching portion 21B and second branching portion 21C at the rear end of the extended portion 21A. Inside the first branching portion 21B, a portion of a first retracting member 4A and a portion of the knot pusher 5 are both inserted. Inside the second branching portion 21C, a portion of a second retracting member 4B is inserted.

The second insertion hole 22A, third insertion hole 22B, fourth insertion hole 23A, fifth insertion hole 23B, sixth insertion hole 24A, seventh insertion hole 24B, and eighth insertion hole 25 illustrated in FIG. 4 extend in the front-rear direction.

The second insertion hole 22A and third insertion hole 22B are arranged in a center of the body 2A in the up-down direction. The second insertion hole 22A is disposed to the right of the first insertion hole 21. The third insertion hole 22B is disposed to the left of the first insertion hole 21. A third control wire 221 is inserted in the second insertion hole 22A and third insertion hole 22B.

The fourth insertion hole 23A and the fifth insertion hole 23B are arranged lower than the second branching portion 21C of the first insertion hole 21. The fourth insertion hole 23A is disposed to the right of the second branching portion 21C. A first control wire 201 and a fourth control wire 231 are inserted in the fourth insertion hole 23A. The fifth insertion hole 23B is disposed to the left of the second branching portion 21C. A fifth control wire 232 is inserted in the fifth insertion hole 23B.

The sixth insertion hole 24A is disposed on the right side of the first branching portion 21B. The first control wire 201 and a second control wire 202 are inserted in the sixth insertion hole 24A. The seventh insertion hole 24B is disposed on the left side of the first branching portion 21B. The second control wire 202 is inserted in the seventh insertion hole 24B. The pivoting part 20B of the body 2A is configured to pivot relative to the base 20A in response to operations on the second control wire 202. The eighth insertion hole 25 is disposed below the second branching portion 21C. A sixth control wire 251 is inserted in the eighth insertion hole 25.

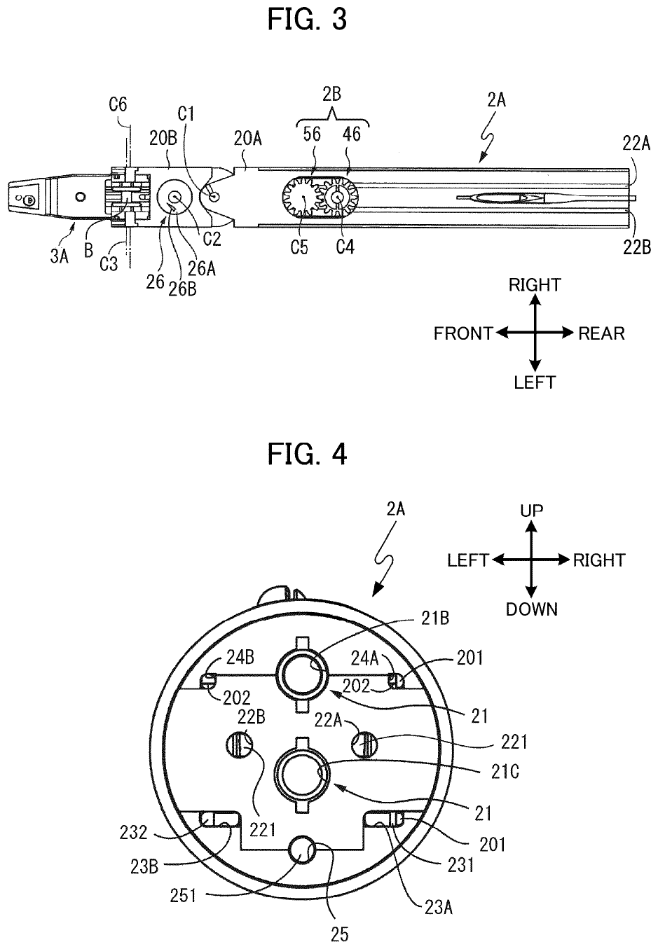

As illustrated in FIG. 2, a first cutter 26 is disposed inside the pivoting part 20B. As illustrated in FIG. 3, the first cutter 26 includes a rotary base 26A and a blade 26B. The rotary base 26A is rotatable about a second axis C2 that extends in the up-down direction. The rotary base 26A is configured to rotate in response to operations on the fourth control wire 231 and fifth control wire 232 (see FIG. 4). The blade 26B extends upward from a top surface of the rotary base 26A. When the rotary base 26A rotates, the blade 26B passes through the extended portion 21A of the first insertion hole 21 (see FIG. 2). By passing through the extended portion 21A, the blade 26B cuts the thread T inside the extended portion 21A.

As illustrated in FIG. 2, a bobbin B is arranged inside the pivoting part 20B at a position frontward of the first cutter 26. The thread Tis wound around the bobbin B. The bobbin B is supported so as to be rotatable about a third axis C3 extending in the left-right direction.

### <Forming Part 2B>

As illustrated in FIGS. 2 and 3, the forming part 2B is disposed inside the body 2A at a position rearward of the first cutter 26. The forming part 2B is configured to form a loop in the thread T within the extended portion 21A of the first insertion hole 21. The forming part 2B includes a first looping shaft 46, and a second looping shaft 56.

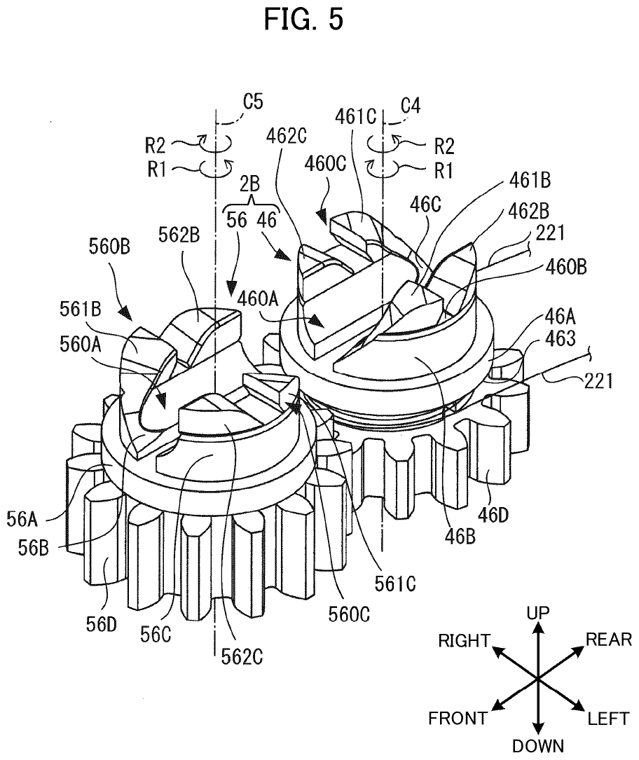

As illustrated in FIG. 5, the first looping shaft 46 has a first support base 46A, a first separated base 46B, a second separated base 46C, a first separated wall 461B, a second separated wall 462B, a third separated wall 461C, a fourth separated wall 462C, and a first gear 46D. The second looping shaft 56 has a second support base 56A, a third separated base 56B, a fourth separated base 56C, a fifth separated wall 561B, a sixth separated wall 562B, a seventh separated wall 561C, an eighth separated wall 562C, and a second gear 56D.

The first support base 46A and the second support base 56A are circular plates and are orthogonal to the up-down direction. The first support base 46A and second support base 56A are aligned in the front-rear direction. The first support base 46A is arranged to the rear of the second support base 56A. The first support base 46A is rotatable about a fourth axis C4 that extends in the up-down direction through a center of the first support base 46A. The second support base 56A is rotatable about a fifth axis C5 that extends in the up-down direction through a center of the second support base 56A.

A first groove 463 is formed in a side surface of the first support base 46A. The third control wire 221 extending forward from the rear of the first support base 46A is wound around the first groove 463, changing a route to extend toward the rear. The first gear 46D is disposed on a bottom of the first support base 46A. The second gear 56D is disposed on a bottom of the second support base 56A. The first gear 46D and the second gear 56D are meshed with each other. The first support base 46A and the second support base 56A are configured to rotate in association with each other in response to operations on the third control wire 221.

When the first support base 46A rotates in a clockwise direction when viewed from above, the second support base 56A rotates in a counterclockwise direction. Hereinafter, these directions of rotation will be called a “first rotating direction R1.” When the first support base 46A rotates in a counterclockwise direction when viewed from above, the second support base 56A rotates in a clockwise direction. Hereinafter, these directions of rotation will be called a “second rotating direction R2.” Unless otherwise specified, “clockwise direction” and “counterclockwise direction” in the following description will denote the directions of rotation seen when viewed from above.

The first separated base 46B and the second separated base 46C are disposed on a top surface of the first support base 46A. The first separated base 46B and second separated base 46C are spaced apart from each other in a radial direction with respect to the fourth axis C4. Specifically, the first separated base 46B and the second separated base 46C are positioned opposite each other with respect to the fourth axis C4. A second groove 460A is formed between the first separated base 46B and second separated base 46C. The third separated base 56B and fourth separated base 56C are disposed on a top surface of the second support base 56A. The third separated base 56B and fourth separated base 56C are spaced apart from each other in a radial direction with respect to the fifth axis C5. Specifically, the third separated base 56B and the fourth separated base 56C are positioned opposite each other with respect to the fifth axis C5. A third groove 560A is formed between the third separated base 56B and fourth separated base 56C.

The first separated wall 461B and second separated wall 462B protrude upward from the first separated base 46B. The first separated wall 461B and second separated wall 462B are spaced apart from each other in a circumferential direction about the fourth axis C4. A fourth groove 460B is formed between the first separated wall 461B and second separated wall 462B. The third separated wall 461C and fourth separated wall 462C protrude upward from the second separated base 46C. The third separated wall 461C and fourth separated wall 462C are spaced apart from each other in the circumferential direction about the fourth axis C4. A fifth groove 460C is formed between the third separated wall 461C and fourth separated wall 462C. Top surfaces of the first separated wall 461B, second separated wall 462B, third separated wall 461C, and fourth separated wall 462C slope upward from their downstream ends in the clockwise direction toward their opposite ends.

The fifth separated wall 561B and sixth separated wall 562B protrude upward from the third separated base 56B. The fifth separated wall 561B and sixth separated wall 562B are spaced apart from each other in a circumferential direction about the fifth axis C5. A sixth groove 560B is formed between the fifth separated wall 561B and sixth separated wall 562B. The seventh separated wall 561C and eighth separated wall 562C protrude upward from the fourth separated base 56C. The seventh separated wall 561C and eighth separated wall 562C are spaced apart from each other in the circumferential direction about the fifth axis C5. A seventh groove 560C is formed between the seventh separated wall 561C and eighth separated wall 562C. Top surfaces of the fifth separated wall 561B, sixth separated wall 562B, seventh separated wall 561C, and eighth separated wall 562C slope upward from their downstream ends in the counterclockwise direction toward their opposite ends.

As illustrated in FIG. 5, when the first looping shaft 46 rotates to position the second separated base 46C on the right side of the first separated base 46B, the second looping shaft 56 also rotates in association with the rotation of the first looping shaft 46 to position the fourth separated base 56C on the left side of the third separated base 56B. Hereinafter, the rotated position illustrated in FIG. 5 will be called a “first rotational position”; the position in which the first looping shaft 46 and second looping shaft 56 have been rotated 90 degrees in the first rotating direction R1 from the first rotational position will be called a “second rotational position”; and the position in which the first looping shaft 46 and second looping shaft 56 have been rotated 180 degrees from the first rotational position will be called a “third rotational position.”

While the first looping shaft 46 and second looping shaft 56 are arranged in the first rotational position or the third rotational position, the second groove 460A and third groove 560A extend in the front-rear direction, and the fourth groove 460B, fifth groove 460C, sixth groove 560B, and seventh groove 560C extend in the left-right direction. While the first looping shaft 46 and second looping shaft 56 are arranged in the second rotational position, the second groove 460A and third groove 560A extend in the left-right direction, and the fourth groove 460B, fifth groove 460C, sixth groove 560B, and seventh groove 560C extend in the front-rear direction.

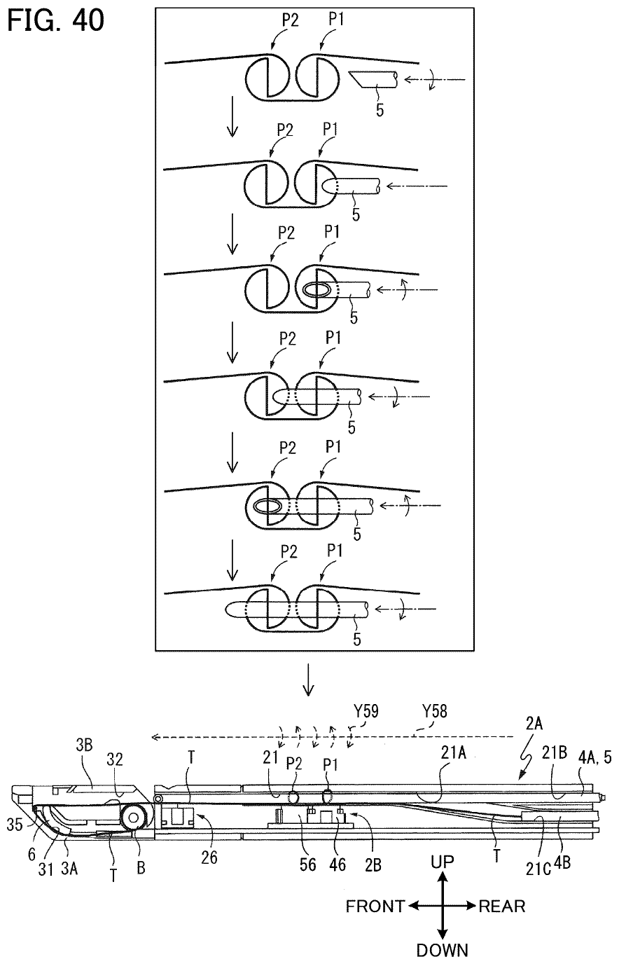

The forming part 2B can form a first loop P1 and a second loop P2 (see FIG. 40) by wrapping the thread T around the first looping shaft 46 and the second looping shaft 56. The first loop P1 is formed on the first looping shaft 46, and the second loop P2 is formed on the second looping shaft 56. Further, by rotating the first looping shaft 46 and second looping shaft 56 while the first loop P1 and second loop P2 are formed, the forming part 2B can remove the first loop P1 and second loop P2 from the first looping shaft 46 and second looping shaft 56 while maintaining the first loop P1 and second loop P2 formed in the thread T.

### <Holder 3>

As illustrated in FIG. 1, the holder 3 is disposed on the front end of the body 2A. The holder 3 includes a first jaw part 3A and a second jaw part 3B. The first jaw part 3A and second jaw part 3B hold the ligation target S.

The first jaw part 3A extends frontward from the front end of the body 2A, and then bends and extends diagonally upward and frontward. The second jaw part 3B has a rear end that is pivotably movably supported on the front end of the body 2A. The second jaw part 3B is pivotable about a sixth axis C6 that extends in the left-right direction. In accordance with the pivoting movement of the second jaw part 3B, a front end of the second jaw part 3B is movable between: an adjacent position (see FIG. 2) in which the front end of the second jaw part 3B contacts a front end of the first jaw part 3A; and a separated position (see FIG. 1) in which the front end of the second jaw part 3B is above and separated from the front end of the first jaw part 3A. The second jaw part 3B is movable between the adjacent position and the separated position in response to operations on the first control wire 201 (see FIG. 4).

While the second jaw part 3B is in the adjacent position, as illustrated in FIG. 2, a gap is formed between a part of the first jaw part 3A, the part excluding another portion of the first jaw part 3A that contacts the second jaw part 3B, and a part of the second jaw part 3B, the part excluding another portion of the second jaw part 3B that contacts the first jaw part 3A. The ligation target S is held by the first jaw part 3A and the second jaw part 3B while being accommodated in this gap.

A first insertion hole 31 is formed inside the first jaw part 3A. The first insertion hole 31 extends diagonally downward and rearward from the portion of the first jaw part 3A (which contacts the second jaw part 3B when the second jaw part 3B is in the adjacent position), and then bends and extends rearward. The feeder 6 described later (see FIG. 17) is disposed in the first insertion hole 31. A latching pin 35 is disposed at a front end of the first insertion hole 31 for anchoring the thread T.

A recess 32 is formed in a bottom surface of the second jaw part 3B. The recess 32 is recessed upward. The recess 32 extends rearward from the front end toward a rear end of the second jaw part 3B. The recess 32 has a rear end that is positioned frontward relative to a front end of the first insertion hole 21 formed in the body 2A. A second insertion hole 33 is formed near the front end of the second jaw part 3B. The second insertion hole 33 extends through the second jaw part 3B in the up-down direction. The recess 32 and the second insertion hole 33 intersect each other. A second cutter 34 is fixed to a surface of the second jaw part 3B constituting a rear end of the second insertion hole 33. The second cutter 34 has a blade facing the inside of the second insertion hole 33.

### <Retractor 4>

At least a part of the retractor 4 is disposed in the body 2A. The retractor 4 is movable in the front-rear direction relative to the body 2A. The retractor 4 is configured to draw the thread T into the body 2A by moving rearward. As illustrated in FIGS. 6, 7, and 8, the retractor 4 includes the first retracting member 4A and the second retracting member 4B. The first retracting member 4A and the second retracting member 4B have the same shape as each other. The first retracting member 4A is arranged above the second retracting member 4B. The first retracting member 4A is disposed inside the knot pusher 5 described later.

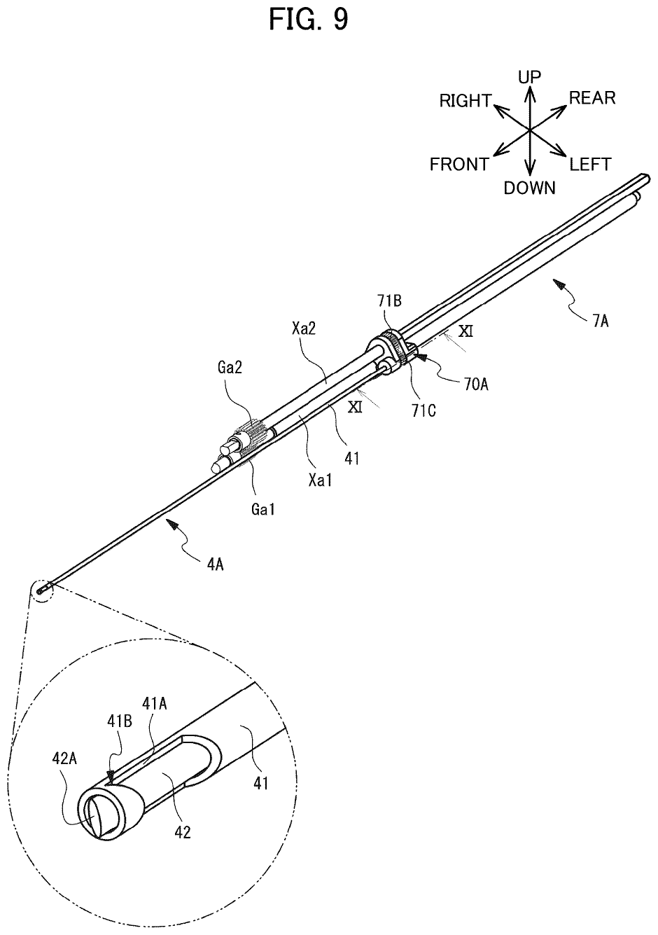

As illustrated in FIG. 9, the first retracting member 4A has a hook body 41, and a wire 42. The hook body 41 has a hollow cylindrical shape and extends in the front-rear direction. A notch 41A is formed in the hook body 41 at a position near a front end of the hook body 41 on the left side thereof. Of peripheral surfaces defining the notch 41A, those surfaces extending rightward from a left edge of the notch 41A slope frontward. In this way, the hook body 41 has a front end portion that is bent into a hook-like shape when viewed from above. This hook-shaped portion of the hook body 41 will be called a “hook 41B.” The hook 41B functions to hook the thread T.

The wire 42 is arranged in a through-hole inside the hook body 41. The wire 42 has a columnar shape and extends in the front-rear direction. The wire 42 has a front surface 42A that slopes relative to a plane orthogonal to the front-rear direction. When viewed from above, the front surface 42A slopes diagonally rearward and rightward from a left edge thereof. The wire 42 functions to hold the thread T hooked by the hook 41B of the hook body 41.

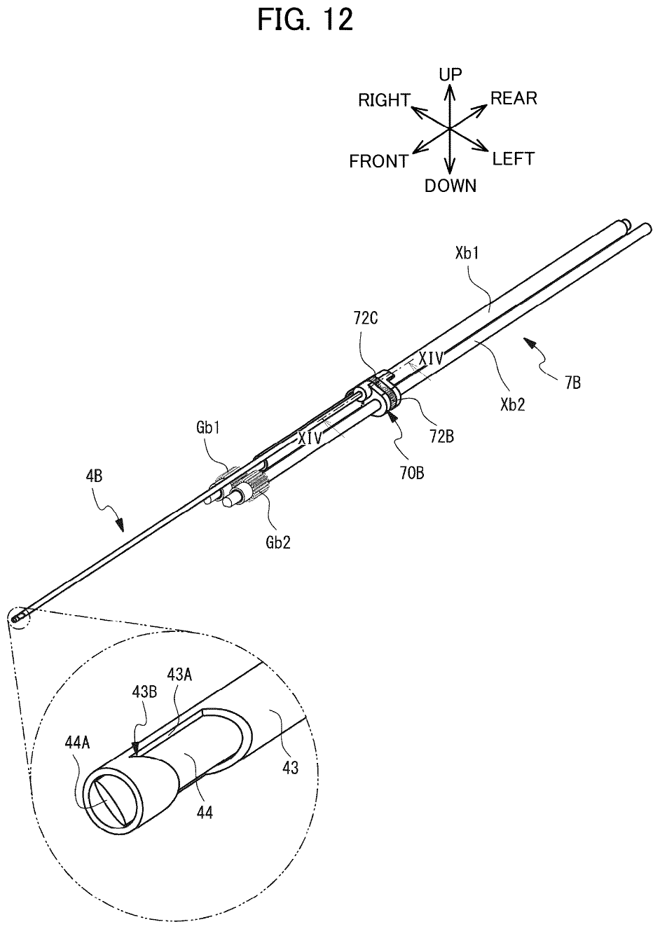

As illustrated in FIG. 12, the second retracting member 4B has a hook body 43, and a wire 44. The hook body 43 and wire 44 correspond to the hook body 41 and wire 42 of the first retracting member 4A (see FIG. 9), respectively. A notch 43A and a hook 43B of the hook body 43 correspond to the notch 41A and the hook 41B of the hook body 41 (see FIG. 9), respectively. A front surface 44A of the wire 44 corresponds to the front surface 42A of the wire 42 (see FIG. 9).

The first retracting member 4A is configured to move in the front-rear direction along the extended portion 21A and the first branching portion 21B of the first insertion hole 21 (see FIG. 2) inside the body 2A and along the recess 32 of the second jaw part 3B (see FIG. 2). The second retracting member 4B is configured to move in the front-rear direction along the extended portion 21A and the second branching portion 21C of the first insertion hole 21 (see FIG. 2) inside the body 2A and along the recess 32 of the second jaw part 3B (see FIG. 2).

### <Knot Pusher 5>

At least a portion of the knot pusher 5 is disposed inside the body 2A. The knot pusher 5 is movable in the front-rear direction relative to the body 2A. By moving forward, the knot pusher 5 is configured to push the first loop P1 and second loop P2 of the thread T formed by the forming part 2B frontward from the body 2A.

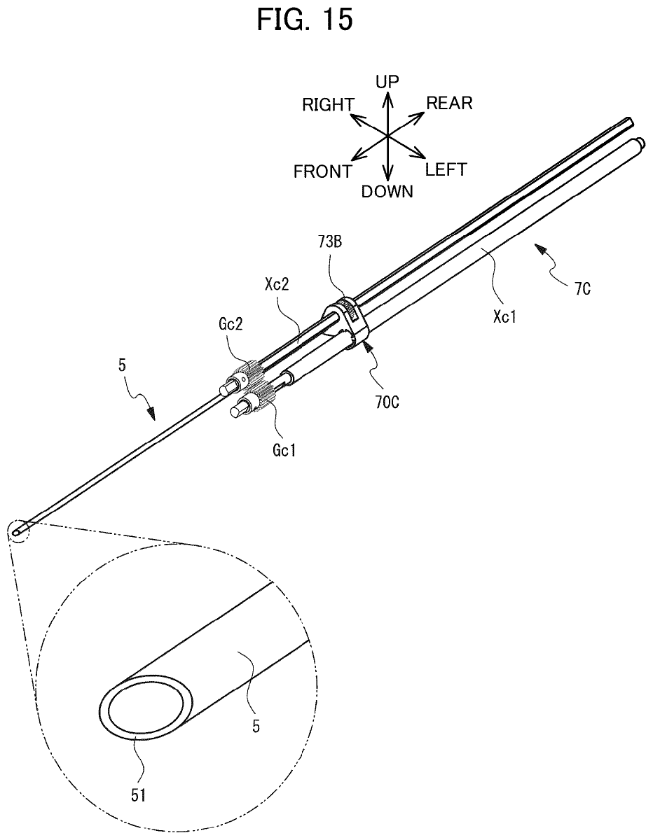

As illustrated in FIG. 15, the knot pusher 5 has a hollow cylindrical shape and extends in the front-rear direction. The first retracting member 4A (see FIG. 9) is disposed inside the knot pusher 5. A centerline extending in the front-rear direction through a center of the knot pusher 5 is coincident with a centerline extending in the front-rear direction through a center of the first retracting member 4A. The knot pusher 5 has a front end 51 that slopes relative to a plane orthogonal to the front-rear direction.

The knot pusher 5 is configured to move in the front-rear direction along the extended portion 21A and the first branching portion 21B of the first insertion hole 21 within the body 2A and along the recess 32 of the second jaw part 3B (see FIG. 2). The knot pusher 5 is also movable in the front-rear direction relative to the first retracting member 4A that is disposed inside the knot pusher 5.

### <Feeder 6>

The feeder 6 illustrated in FIG. 17 is arranged inside the first jaw part 3A of the holder 3 (see FIG. 1). The feeder 6 is movable in the front-rear direction along the first insertion hole 31 (see FIG. 2). The feeder 6 is configured to feed the thread T from the first jaw part 3A toward the second jaw part 3B. The feeder 6 has a base 61, a groove 62, a coupling part 63, and a connector 64.

The base 61 has a shape of a column curved into an arc. The base 61 extends upward toward a front end thereof. The groove 62 is arranged in the front end of the base 61. The groove 62 extends in the front-rear direction. The groove 62 is positioned farther rightward than a left-right center of the base 61. The groove 62 functions to hold the thread T between side surfaces of the base 61 defining the groove 62. A notch 60 is formed in a right surface of the base 61. The notch 60 has a bottom surface that is positioned farther leftward than the groove 62.

The coupling part 63 extends rearward from a rear end of the base 61. The coupling part 63 has a columnar shape with a smaller cross-sectional diameter than the base 61. The coupling part 63 is flexible. The connector 64 is disposed on a rear end of the coupling part 63. The sixth control wire 251 (see FIG. 4) is connected to the connector 64. The feeder 6 is configured to move in the front-rear direction in response to operations on the sixth control wire 251.

### <Robot Connector 9>

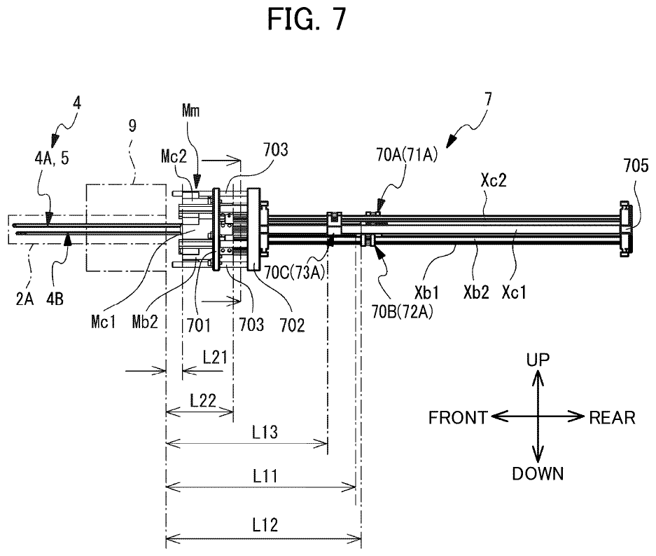

As illustrated in FIG. 1, the robot connector 9 covers a rear end of the body 2A. The robot connector 9 has a cylindrical shape. The robot connector 9 has a closed bottom end in which an opening at the bottom is covered by a bottom surface, and a side surface 91. The body 2A passes rearward through the side surface 91 at a front portion thereof, and extends to a rear end of the side surface 91. The retractor 4 and the knot pusher 5 pass through the body 2A and extend farther rearward than the rear end of the side surface 91 (see FIG. 7).

The robot connector 9 has a top end formed with an opening 9A. A disk Rr of the robot R is fitted in the opening 9A at the top end of the robot connector 9, whereby the robot connector 9 is connected to the robot R. A plurality of robot motors MR built in the robot R is connected to the disk Rr. Rotational shafts of the robot motors MR extend into the robot connector 9. The robot R is configured to operate the first control wire 201, second control wire 202, third control wire 221, fourth control wire 231, fifth control wire 232, and sixth control wire 251 (see FIG. 4) by rotating the robot motors MR.

### <Drive Unit 7>

As illustrated in FIG. 1, the drive unit 7 is connected to the robot connector 9 at the rear end of the side surface 91. The drive unit 7 includes a first drive mechanism 7A (see FIG. 9), a second drive mechanism 7B (see FIG. 12), a third drive mechanism 7C (see FIG. 15), a first support plate 701, a second support plate 702, a plurality of spacers 703, a case 704, and a base plate 705.

The first support plate 701 and second support plate 702 are circular plates and are orthogonal to the front-rear direction. The spacers 703 are disposed between the first support plate 701 and second support plate 702. The spacers 703 support the first support plate 701 and second support plate 702 such that the first support plate 701 and second support plate 702 are spaced apart from each other in the front-rear direction. The second support plate 702 is arranged to the rear of the first support plate 701. The case 704 has a hollow cylindrical shape. The case 704 defines a central axis extending in the front-rear direction. The case 704 has a front end connected to a rear surface of the second support plate 702, and a rear end that is closed by the base plate 705.

The first drive mechanism 7A illustrated in FIGS. 9, 10, and 11 is configured to move the hook body 41 and wire 42 of the first retracting member 4A in the front-rear direction. The second drive mechanism 7B illustrated in FIGS. 12, 13, and 14 is configured to move the hook body 43 and wire 44 of the second retracting member 4B in the front-rear direction. The third drive mechanism 7C illustrated in FIGS. 15 and 16 is configured to move the knot pusher 5 in the front-rear direction and rotate the same. Configurations of the first drive mechanism 7A, second drive mechanism 7B, and third drive mechanism 7C will be described later in detail.

As illustrated in FIG. 6, a first motor Ma1, a first auxiliary motor Ma2, a first motor Mb1, a first auxiliary motor Mb2, a second motor Mc1, and a second auxiliary motor Mc2 (hereinafter collectively referred to as “drive motors Mm”) are fixed to a front surface of the first support plate 701. Respective rotational shafts of the drive motors Mm pass rearward through corresponding through-holes in the first support plate 701 and protrude rearward from a rear surface of the first support plate 701.

As illustrated in FIGS. 6 and 7, first threaded shafts Xa1 and Xb1 and a second threaded shaft Xc1 (hereinafter collectively referred to as the “threaded shafts X1”), and first auxiliary shafts Xa2 and Xb2 and a second auxiliary shaft Xc2 (hereinafter collectively referred to as the “auxiliary shafts X2”) extend in the front-rear direction to span between the second support plate 702 and the base plate 705. The threaded shafts X1 have rod shapes with circular cross sections. Male threads are formed on respective peripheral surfaces of the threaded shafts X1. The male threads are spiral shaped and extend in the front-rear direction. The auxiliary shafts X2 are D-cut shafts each having a D-shaped cross section.

Respective front ends of the threaded shafts X1 and auxiliary shafts X2 pass forward through corresponding through-holes formed in the second support plate 702 and protrude forward from a front surface of the second support plate 702. The threaded shafts X1 and auxiliary shafts X2 are rotatably supported by the second support plate 702 and the base plate 705. The threaded shafts X1 and auxiliary shafts X2, as well as a first hook support part 70A, a second hook support part 70B, and a pusher support part 70C described later, are covered by the case 704.

### <First Drive Mechanism 7A>

As illustrated in FIG. 9, the first drive mechanism 7A includes the first threaded shaft Xa1, the first auxiliary shaft Xa2, the first hook support part 70A, the first motor Ma1, and the first auxiliary motor Ma2 (see FIG. 6).

The first threaded shaft Xa1 is arranged diagonally downward and rightward from the first auxiliary shaft Xa2, and extends parallel to the first auxiliary shaft Xa2. A first gear Ga1 is connected to a portion of the first threaded shaft Xa1 that protrudes forward from the second support plate 702 (see FIG. 6). The first gear Ga1 meshes with a gear connected to the rotational shaft of the first motor Ma1 (see FIG. 6). The first threaded shaft Xa1 is configured to rotate when the first motor Ma1 is driven. A second gear Ga2 is connected to a portion of the first auxiliary shaft Xa2 that protrudes forward from the second support plate 702. The second gear Ga2 meshes with a gear connected to the rotational shaft of the first auxiliary motor Ma2 (see FIG. 6). The first auxiliary shaft Xa2 is configured to rotate when the first auxiliary motor Ma2 is driven.

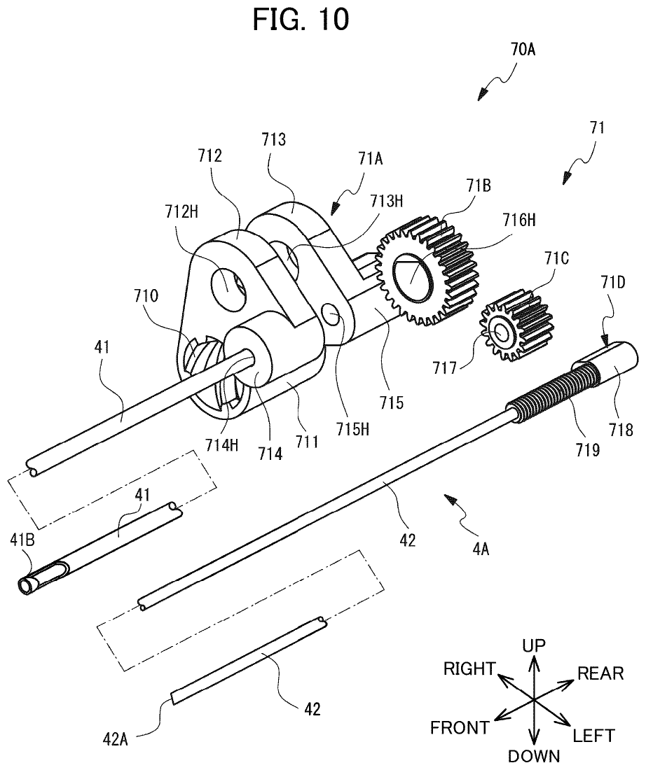

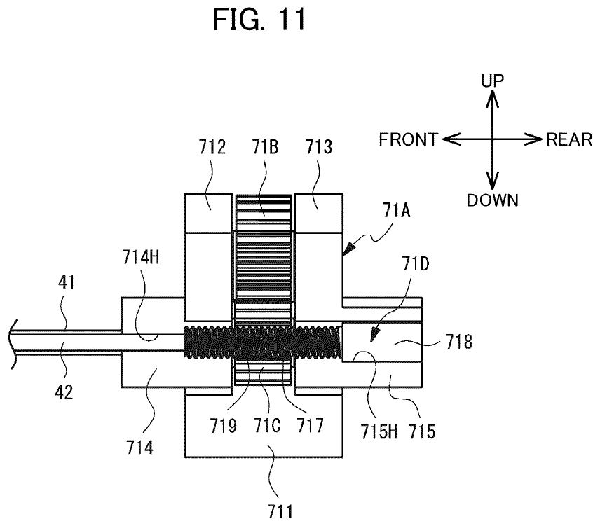

The first hook support part 70A supports the hook body 41 and wire 42. As illustrated in FIGS. 10 and 11, the first hook support part 70A has a support base 71A, a first main gear 71B, a first follow gear 71C, and a first converter 71D.

The support base 71A supports the hook body 41, first main gear 71B, first follow gear 71C, and first converter 71D. The support base 71A has a base 711, a first holding part 712, a second holding part 713, a first protruding part 714, and a second protruding part 715. The base 711 has a cylindrical shape. The base 711 defines a central axis extending in the front-rear direction. A through-hole extending in the front-rear direction is formed in the base 711. A female thread is formed in an inner surface of the through-hole. The female thread has a spiral shape that extends in the front-rear direction. The through-hole having this female thread formed on its inner surface will be called a “first nut portion 710.” The first threaded shaft Xa1 (see FIG. 9) is inserted into the first nut portion 710. The male thread on the first threaded shaft Xa1 meshes with the female thread in the first nut portion 710.

The first holding part 712 and second holding part 713 protrude diagonally upward and leftward from a side surface of the base 711. The first holding part 712 and second holding part 713 have plate shapes and are orthogonal to the front-rear direction. The first holding part 712 and second holding part 713 are spaced apart from each other in the front-rear direction. The first holding part 712 is arranged frontward of the second holding part 713. A first through-hole 712H is formed in an upper end portion of the first holding part 712 and extends through the first holding part 712 in the front-rear direction. A second through-hole 713H is formed in an upper end portion of the second holding part 713 and extends through the second holding part 713 in the front-rear direction. The first through-hole 712H and second through-hole 713H are arranged in line in the front-rear direction. The first auxiliary shaft Xa2 (see FIG. 9) is inserted in the first through-hole 712H and second through-hole 713H.

The first protruding part 714 protrudes frontward from a front surface of the first holding part 712 at a left end thereof. A first through-hole 714H is formed in the first holding part 712 and the first protruding part 714 to extend through the same in the front-rear direction. A rear end of the hook body 41 is connected to a front surface of the first protruding part 714 around the first through-hole 714H (see FIG. 11). The second protruding part 715 protrudes rearward from a rear surface of the second holding part 713 at a left end thereof. A second through-hole 715H is formed in the second holding part 713 and the second protruding part 715 to extend through the same in the front-rear direction. The first through-hole 714H and second through-hole 715H are arranged in line in the front-rear direction.

The first main gear 71B is a spur gear. The first main gear 71B defines a rotational axis extending in the front-rear direction. The first main gear 71B is arranged between the upper end portion of the first holding part 712 and the upper end portion of the second holding part 713. A gear through-hole 716H is formed in a center of the first main gear 71B. The gear through-hole 716H extends in the front-rear direction, and has a D-shaped cross section. The first through-hole 712H in the first holding part 712, the second through-hole 713H in the second holding part 713, and the gear through-hole 716H in the first main gear 71B are arranged in line in the front-rear direction. The first auxiliary shaft Xa2 (see FIG. 9) is inserted in the gear through-hole 716H. The first main gear 71B is movable in the front-rear direction relative to the first auxiliary shaft Xa2. Note that the first auxiliary shaft Xa2 has a D-shaped cross section. Accordingly, the first main gear 71B is configured to rotate in accordance with the rotation of the first auxiliary shaft Xa2.

The first follow gear 71C is a spur gear. The first follow gear 71C defines a rotational axis extending in the front-rear direction. The first follow gear 71C is arranged between left end portions of the first holding part 712 and second holding part 713. A through-hole extending in the front-rear direction is formed in the first follow gear 71C to extend through a center thereof. A female thread is formed on an inner surface of the through-hole. The female thread has a spiral shape that extends in the front-rear direction. As illustrated in FIG. 11, the through-hole having this female thread formed on the inner surface thereof will be called a “nut portion 717.” The first through-hole 714H formed in the first holding part 712 and first protruding part 714, the second through-hole 715H formed in the second holding part 713 and second protruding part 715, and the nut portion 717 are arranged in line in the front-rear direction.

The first follow gear 71C meshes with the first main gear 71B. Accordingly, the first follow gear 71C is configured to rotate in accordance with the rotation of the first main gear 71B.

As illustrated in FIGS. 10 and 11, the first converter 71D is disposed on a rear end of the wire 42. The first converter 71D has an end portion 718 and a follow gear 719. The end portion 718 is connected to the rear end of the wire 42. The end portion 718 is arranged inside the second through-hole 715H. The end portion 718 is movable in the front-rear direction along the second through-hole 715H. The wire 42 extends frontward from the end portion 718. The wire 42 passes through the second through-hole 715H, the nut portion 717 in the first follow gear 71C, and the first through-hole 714H and is inserted in the through-hole in the hook body 41.

The follow gear 719 is connected to a circumference of the wire 42 in front of the end portion 718. A male thread is formed on a side surface of the follow gear 719. The male thread of the follow gear 719 meshes with the female thread in the nut portion 717.

### <Second Drive Mechanism 7B>

As illustrated in FIG. 12, the second drive mechanism 7B includes the first threaded shaft Xb1, the first auxiliary shaft Xb2, the second hook support part 70B, the first motor Mb1, and the first auxiliary motor Mb2 (see FIG. 6).

The first threaded shaft Xb1 is arranged to the right of the first auxiliary shaft Xb2 and extends parallel to the first auxiliary shaft Xb2. A third gear Gb1 is connected to a portion of the first threaded shaft Xb1 that protrudes forward from the second support plate 702 (see FIG. 6). The third gear Gb1 meshes with a gear connected to the rotational shaft of the first motor Mb1 (see FIG. 6). The first threaded shaft Xb1 is configured to rotate when the first motor Mb1 is driven. A fourth gear Gb2 is connected to a portion of the first auxiliary shaft Xb2 that protrudes forward from the second support plate 702. The fourth gear Gb2 meshes with a gear connected to the rotational shaft of the first auxiliary motor Mb2 (see FIG. 6). The first auxiliary shaft Xb2 is configured to rotate when the first auxiliary motor Mb2 is driven.

The second hook support part 70B supports the hook body 43 and the wire 44. The second hook support part 70B has a similar configuration to the first hook support part 70A (see FIGS. 10 and 11). Below, descriptions of parts having common configurations with the first hook support part 70A will be omitted or simplified.

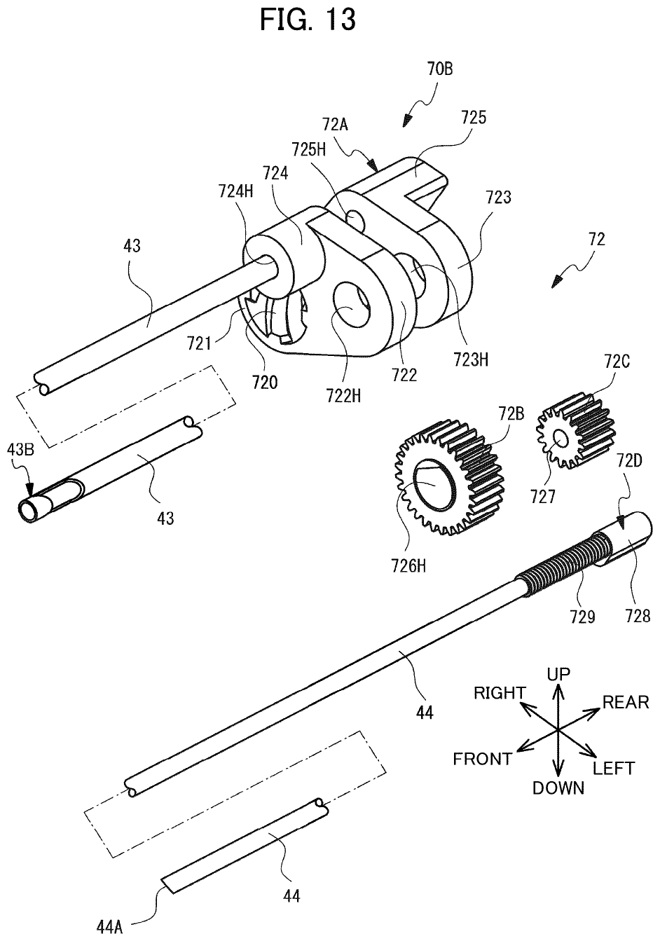

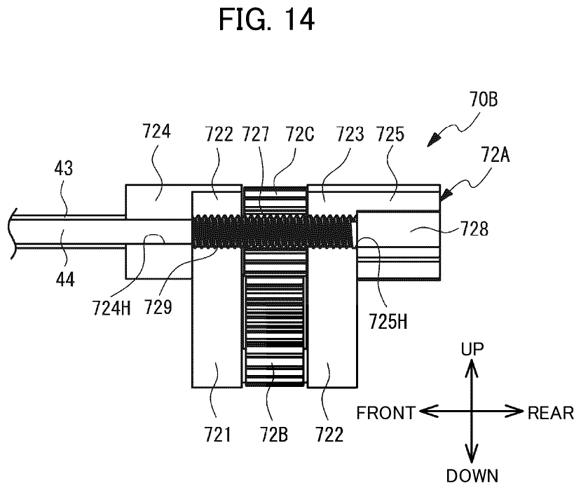

As illustrated in FIGS. 13 and 14, the second hook support part 70B has a support base 72A, a first main gear 72B, a first follow gear 72C, and a first converter 72D. The support base 72A, first main gear 72B, first follow gear 72C, and first converter 72D correspond to the support base 71A, first main gear 71B, first follow gear 71C, and first converter 71D of the first hook support part 70A (see FIGS. 10 and 11), respectively. The support base 72A has a base 721, a first holding part 722, a second holding part 723, a first protruding part 724, and a second protruding part 725 corresponding to the base 711, first holding part 712, second holding part 713, first protruding part 714, and second protruding part 715 of the support base 71A (see FIGS. 10 and 11), respectively. A first nut portion 720 formed in the base 721 corresponds to the first nut portion 710 formed in the base 711 of the support base 71A (see FIG. 10).

The first threaded shaft Xb1 is inserted in the first nut portion 720 (see FIG. 12). The male thread on the first threaded shaft Xb1 meshes with the female thread in the first nut portion 720.

The first holding part 722 and second holding part 723 protrude diagonally upward and leftward from the side surface of the base 721. A first through-hole 722H formed in a left end portion of the first holding part 722 and a second through-hole 723H formed in a left end portion of the second holding part 723 correspond to the first through-hole 712H and second through-hole 713H of the support base 71A (see FIG. 10), respectively. The first through-hole 722H and second through-hole 723H are arranged in line in the front-rear direction. The first auxiliary shaft Xb2 (see FIG. 12) is inserted in the first through-hole 722H and the second through-hole 723H.

The first protruding part 724 protrudes frontward from a front surface of the first holding part 722 at an upper end thereof. A first through-hole 724H formed in the first holding part 722 and first protruding part 724 corresponds to the first through-hole 714H of the support base 71A (see FIGS. 10 and 11). A rear end of the hook body 43 is connected to a front surface of the first protruding part 724 around the periphery of the first through-hole 724H (see FIG. 14). The second protruding part 725 protrudes rearward from a rear surface of the second holding part 723 at an upper end thereof. A second through-hole 725H formed in the second holding part 723 and second protruding part 725 corresponds to the second through-hole 715H of the support base 71A (see FIGS. 10 and 11). The first through-hole 724H and second through-hole 725H are arranged in line in the front-rear direction.

A gear through-hole 726H in the first main gear 72B corresponds to the gear through-hole 716H in the first main gear 71B of the first hook support part 70A (see FIGS. 10 and 11). The first through-hole 722H in the first holding part 722, the second through-hole 723H in the second holding part 723, and the gear through-hole 726H in the first main gear 72B are arranged in line in the front-rear direction. The first auxiliary shaft Xb2 (see FIG. 12) is inserted in the gear through-hole 726H. The first main gear 72B is movable in the front-rear direction relative to the first auxiliary shaft Xb2. The first main gear 72B is configured to rotate in accordance with the rotation of the first auxiliary shaft Xb2.

The first follow gear 72C is arranged between upper end portions of the first holding part 722 and the second holding part 723. A through-hole is formed in the first follow gear 72C, and a female thread is formed in an inner surface of the through-hole. The through-hole having this female thread on its inner surface will be called a “nut portion 727.” As illustrated in FIG. 14, the first through-hole 724H formed in the first holding part 722 and first protruding part 724, the second through-hole 725H formed in the second holding part 723 and second protruding part 725, and the nut portion 727 are arranged in line in the front-rear direction. The first follow gear 72C meshes with the first main gear 72B. Accordingly, the first follow gear 72C is rotatable in accordance with the rotation of the first main gear 72B.

As illustrated in FIGS. 13 and 14, the first converter 72D has an end portion 728 and a follow gear 729 respectively corresponding to the end portion 718 and follow gear 719 of the first converter 71D in the first hook support part 70A (see FIGS. 10 and 11). The wire 44 extends frontward from the end portion 728. The wire 44 passes through the second through-hole 725H, the nut portion 727 of the first follow gear 72C, and the first through-hole 724H, and is inserted in the through-hole in the hook body 43. The male thread on the follow gear 729 meshes with the female thread in the nut portion 727.

### <Third Drive Mechanism 7C>

As illustrated in FIG. 15, the third drive mechanism 7C includes the second threaded shaft Xc1, the second auxiliary shaft Xc2, the pusher support part 70C, the second motor Mc1, and the second auxiliary motor Mc2 (see FIG. 6).

The second threaded shaft Xc1 is positioned diagonally downward and leftward from the second auxiliary shaft Xc2, and extends parallel to the second auxiliary shaft Xc2. A fifth gear Gc1 is connected to a portion of the second threaded shaft Xc1 that protrudes forward from the second support plate 702 (see FIG. 6). The fifth gear Gc1 meshes with a gear connected to the rotational shaft of the second motor Mc1 (see FIG. 6). The second threaded shaft Xc1 is configured to rotate when the second motor Mc1 is driven. A sixth gear Gc2 is connected to a portion of the second auxiliary shaft Xc2 that protrudes forward from the second support plate 702. The sixth gear Gc2 meshes with a gear connected to the rotational shaft of the second auxiliary motor Mc2 (see FIG. 6). The second auxiliary shaft Xc2 is configured to rotate when the second auxiliary motor Mc2 is driven.

The pusher support part 70C supports the knot pusher 5. With some exceptions, the configuration of the pusher support part 70C is the same as the first hook support part 70A (see FIGS. 10 and 11) and the second hook support part 70B (see FIGS. 13 and 14). Hereinafter, descriptions of parts having common configurations to the first hook support part 70A will be omitted or simplified.

As illustrated in FIG. 16, the pusher support part 70C has a support base 73A, a second main gear 73B, and a second follow gear 739. The support base 73A and second main gear 73B correspond to the support base 71A and first main gear 71B of the first hook support part 70A (see FIG. 10), respectively. The pusher support part 70C has no member that corresponds to the first follow gear 71C of the first hook support part 70A (see FIG. 10). The support base 73A has a base 731, a first holding part 732, and a second holding part 733 that correspond to the base 711, first holding part 712, and second holding part 713 of the support base 71A (see FIGS. 10 and 11). The support base 73A has no parts that correspond to the first protruding part 714 and second protruding part 715 of the first hook support part 70A (see FIGS. 10 and 11). A second nut portion 730 formed in the base 731 corresponds to the first nut portion 710 formed in the base 711 of the support base 71A (see FIG. 10). The second threaded shaft Xc1 (see FIG. 15) is inserted in the second nut portion 730. The male thread on the second threaded shaft Xc1 meshes with the female thread in the second nut portion 730.

The first holding part 732 and second holding part 733 protrude diagonally upward and rightward from a side surface of the base 731. A first through-hole 732H formed in an upper end portion of the first holding part 732 and a second through-hole 733H formed in an upper end portion of the second holding part 733 correspond to the first through-hole 712H and second through-hole 713H of the support base 71A (see FIG. 10), respectively. The first through-hole 732H and the second through-hole 733H are arranged in line in the front-rear direction. The second auxiliary shaft Xc2 (see FIG. 15) is inserted in the first through-hole 732H and the second through-hole 733H.

A first through-hole 734H is formed in a right end portion of the first holding part 732. A second through-hole 735H is formed in a right end portion of the second holding part 733. The first through-hole 734H and second through-hole 735H are arranged in line in the front-rear direction. A rear end portion of the knot pusher 5 is inserted in the first through-hole 734H and second through-hole 735H.

A gear through-hole 736H in the second main gear 73B corresponds to the gear through-hole 716H in the first main gear 71B of the first hook support part 70A (see FIGS. 10 and 11). The first through-hole 732H in the first holding part 732, the second through-hole 733H in the second holding part 733, and the gear through-hole 736H in the second main gear 73B are all arranged in line in the front-rear direction. The second auxiliary shaft Xc2 (see FIG. 12) is inserted in the gear through-hole 736H. The second main gear 73B is movable in the front-rear direction relative to the second auxiliary shaft Xc2. The second main gear 73B is configured to rotate in accordance with the rotation of the second auxiliary shaft Xc2.

The second follow gear 739 is connected to a circumferential portion of the knot pusher 5 that is positioned slightly forward from the rear end of the knot pusher 5. The second follow gear 739 is arranged between the first holding part 732 and second holding part 733. The second follow gear 739 meshes with the second main gear 73B. The second main gear 73B and the second follow gear 739 are configured to rotate in accordance with the rotation of the second auxiliary shaft Xc2.

### <Operations of the First Drive Mechanism 7A>

In the following description, the direction in which the threaded shafts X1 are rotated for moving the hook bodies 41 and 43 and the knot pusher 5 forward will be called a “forward direction.” The direction in which the threaded shafts X1 are rotated for moving the hook bodies 41 and 43 and the knot pusher 5 rearward will be called a “reverse direction.” Further, the direction in which the auxiliary shafts X2 are rotated for moving the wires 42 and 44 forward will be called a “forward direction.” The direction in which the auxiliary shafts X2 are rotated for moving the wires 42 and 44 rearward will be called a “reverse direction.”

For moving the hook body 41 and wire 42 of the first retracting member 4A in FIGS. 9 and 10 forward, the ligation device 1A drives the first motor Ma1 to rotate the first threaded shaft Xa1 in the forward direction. The first nut portion 710 receives a forward force in response to the rotation of the first threaded shaft Xa1 and moves forward. In this case, the support base 71A in which the first nut portion 710 is formed moves forward in response to the movement of the first nut portion 710. As a result, the hook body 41 connected to the first protruding part 714 also moves forward. The first main gear 71B moves forward along the first auxiliary shaft Xa2.

In response to the forward movement of the support base 71A, the wire 42 connected to the end portion 718 disposed in the second through-hole 715H also moves forward. Accordingly, the hook body 41 and wire 42 of the first retracting member 4A move forward together.

When moving the hook body 41 and wire 42 of the first retracting member 4A rearward, on the other hand, the ligation device 1A drives the first motor Ma1 to rotate the first threaded shaft Xa1 in the reverse direction. As a result, the hook body 41 and wire 42 of the first retracting member 4A move rearward together.

Further, in order to move the wire 42 of the first retracting member 4A forward relative to the hook body 41, the ligation device 1A drives the first auxiliary motor Ma2 to rotate the first auxiliary shaft Xa2 (see FIG. 9) in the forward direction. The first main gear 71B and first follow gear 71C rotate in response to the rotation of the first auxiliary shaft Xa2. The follow gear 719 of the first converter 71D receives a forward force from the first follow gear 71C as the first follow gear 71C rotates, and moves forward. In this case, the wire 42 connected to the follow gear 719 also moves forward. Note that the hook body 41 connected to the first protruding part 714 does not move. Accordingly, the wire 42 of the first retracting member 4A moves forward relative to the hook body 41.

When moving the wire 42 rearward relative to the hook body 41, on the other hand, the ligation device 1A drives the first auxiliary motor Ma2 to rotate the first auxiliary shaft Xa2 in the reverse direction. In this case, only the wire 42 moves rearward, while the hook body 41 connected to the first protruding part 714 does not move.

As described above, the first drive mechanism 7A is configured to move the hook body 41 and wire 42 of the first retracting member 4A together in the front-rear direction by driving the first motor Ma1 to rotate the first threaded shaft Xa1. Additionally, the first auxiliary motor Ma2, first auxiliary shaft Xa2, first main gear 71B, first follow gear 71C, and first converter 71D of the first drive mechanism 7A function as a moving mechanism 71 for moving the wire 42 relative to the hook body 41 in the front-rear direction. The first converter 71D is configured to move the wire 42 in the front-rear direction by converting the rotational motion of the first auxiliary shaft Xa2, first main gear 71B, and first follow gear 71C into linear motion.

### <Operations of the Second Drive Mechanism 7B>

The operations of the second drive mechanism 7B for moving the hook body 43 and the wire 44 of the second retracting member 4B in the front-rear direction are the same as the operations of the first drive mechanism 7A. The second drive mechanism 7B in FIGS. 12 and 13 is configured to move the hook body 43 and the wire 44 of the second retracting member 4B together in the front-rear direction by driving the first motor Mb1 to rotate the first threaded shaft Xb1. Further, the first auxiliary motor Mb2, first auxiliary shaft Xb2, first main gear 72B, first follow gear 72C, and first converter 72D of the second drive mechanism 7B function as a moving mechanism 72 for moving the wire 44 in the front-rear direction relative to the hook body 43. The first converter 72D is configured to move the wire 44 in the front-rear direction by converting the rotational motion of the first main gear 72B and first follow gear 72C into linear motion.

### <Operations of the Third Drive Mechanism 7C>

To move the knot pusher 5 in FIGS. 15 and 16 forward, the ligation device 1A drives the second motor Mc1 to rotate the second threaded shaft Xc1 in the forward direction. The second nut portion 730 receives a forward force in response to the rotation of the second threaded shaft Xc1, and moves forward. In this case, the support base 73A in which the second nut portion 730 is formed moves forward in response to the movement of the second nut portion 730. The second follow gear 739 interposed between the first holding part 732 and second holding part 733 of the support base 73A also moves forward. Therefore, the knot pusher 5 whose rear end is connected to the second follow gear 739 moves forward. Note that the second main gear 73B moves forward along the second auxiliary shaft Xc2.

When moving the knot pusher 5 rearward, the ligation device 1A drives the second motor Mc1 to rotate the second threaded shaft Xc1 in the reverse direction.

To rotate the knot pusher 5, the ligation device 1A drives the second auxiliary motor Mc2 to rotate the second auxiliary shaft Xc2. The second main gear 73B and second follow gear 739 rotate in response to the rotation of the second auxiliary shaft Xc2. As a result, the knot pusher 5 connected to the second follow gear 739 rotates about a seventh axis C7, which extends in the front-rear direction through a center of the knot pusher 5. Note that the direction in which the knot pusher 5 rotates when the second auxiliary shaft Xc2 rotates in the forward direction is different from the direction in which the knot pusher 5 rotates when the second auxiliary shaft Xc2 rotates in the reverse direction.

As described above, the third drive mechanism 7C is configured to move the knot pusher 5 in the front-rear direction by driving the second motor Mc1 to rotate the second threaded shaft Xc1. Additionally, the second auxiliary motor Mc2, second auxiliary shaft Xc2, second main gear 73B, and second follow gear 739 of the third drive mechanism 7C function as a rotating mechanism 73 for rotating the knot pusher 5 about the seventh axis C7.

### <Positional Relationships Among the First Drive Mechanism 7A, Second Drive Mechanism 7B, and Third Drive Mechanism 7C>

Referring to FIG. 7, L11 denotes a distance in the front-rear direction between the rear end of the robot connector 9 and the front end of the first hook support part 70A. L12 denotes a distance in the front-rear direction between the rear end of the robot connector 9 and the front end of the second hook support part 70B. L13 denotes a distance in the front-rear direction between the rear end of the robot connector 9 and the front end of the pusher support part 70C. Further, L21 denotes a distance in the front-rear direction between the rear end of the robot connector 9 and the front end of the drive motors Mm. L22 denotes a distance in the front-rear direction between the rear end of the robot connector 9 and the front ends of the threaded shafts X1 and auxiliary shafts X2. Note that the rear end of the robot connector 9 is at the same position in the front-rear direction as the rear end of the body 2A.

As illustrated in FIG. 7, the distance L21 is shorter than the distance L22. In other words, the drive motors Mm are closer to the robot connector 9 than the threaded shafts X1 and auxiliary shafts X2 are to the robot connector 9. The distance L21 is also shorter than the distances L11, L12, and L13. Hence, the drive motors Mm are closer to the robot connector 9 than each of the first hook support part 70A, second hook support part 70B, and pusher support part 70C is to the robot connector 9.

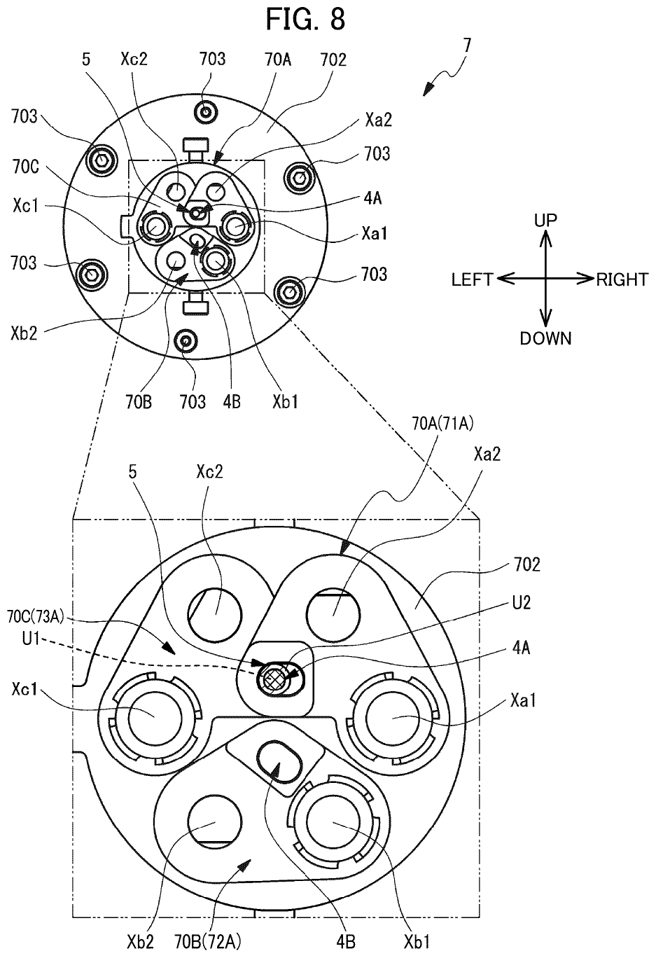

As illustrated in FIG. 8, the positions of the first hook support part 70A (support base 71A) and second hook support part 70B (support base 72A) are mutually different in directions orthogonal to the front-rear direction (i.e., in the up-down and left-right directions). Accordingly, the first hook support part 70A and second hook support part 70B do not contact each other when moving in the front-rear direction. Therefore, the first hook support part 70A can move farther forward than the second hook support part 70B, and the second hook support part 70B can move farther forward than the first hook support part 70A. Even in a state where a part of the first hook support part 70A and a part of the second hook support part 70B overlap each other in position in the front-rear direction as illustrated in FIG. 7, the first hook support part 70A and second hook support part 70B do not contact each other.

When viewed from the rear, the first retracting member 4A and the knot pusher 5 are arranged concentrically, as illustrated in FIG. 8. A portion of the first hook support part 70A (support base 71A) to which the first retracting member 4A is connected and a portion of the pusher support part 70C (support base 73A) to which the knot pusher 5 is connected are at the same position as each other in directions orthogonal to the front-rear direction (i.e., in the up-down and left-right directions). The portion of the first hook support part 70A (support base 71A) to which the first retracting member 4A is connected and the portion of the pusher support part 70C (support base 73A) to which the knot pusher 5 is connected overlap each other in the front-rear direction (i.e., when viewed in the front-rear direction). More specifically, an area U1 enclosed by the first retracting member 4A is contained in an area U2 enclosed by the knot pusher 5 when viewed from the rear.

A portion of the first hook support part 70A (support base 71A) and a portion of the pusher support part 70C (support base 73A) overlap each other in position in directions orthogonal to the front-rear direction (i.e., in the up-down and left-right directions). As illustrated in FIG. 7, the pusher support part 70C is arranged forward of the first hook support part 70A. Thus, movement of the first hook support part 70A toward the front of the pusher support part 70C is restricted by the rear surface of the pusher support part 70C contacting the front surface of the first hook support part 70A. Hence, the first hook support part 70A is always positioned rearward of the pusher support part 70C. The distance L13 is shorter than the distance L11.

On the other hand, the positions of the second hook support part 70B (support base 72A) and the pusher support part 70C (support base 73A) are different in directions orthogonal to the front-rear direction (i.e., in the up-down and left-right directions), as illustrated in FIG. 8. When viewed from the rear, the second hook support part 70B (support base 72A) and pusher support part 70C (support base 73A) do not overlap each other. Therefore, the second hook support part 70B and pusher support part 70C do not contact each other when either moves in the front-rear direction. Accordingly, the second hook support part 70B can move farther forward than the pusher support part 70C, and the pusher support part 70C can move farther forward than the second hook support part 70B. When the pusher support part 70C is positioned forward of the second hook support part 70B, the distance L13 is shorter than the distance L12. However, when the pusher support part 70C is positioned farther rearward than the second hook support part 70B, the distance L13 is longer than the distance L12.

### <Description of a Ligation Process>

A ligation process in which the ligation device 1A ligates the ligation target S with the thread T, will be described. The ligation device 1A starts this ligation process when in an initial state. The initial state is as follows.

The second jaw part 3B is disposed in the adjacent position. The feeder 6 is accommodated in the first insertion hole 31 of the first jaw part 3A. The first retracting member 4A and knot pusher 5 are arranged in the extended portion 21A and first branching portion 21B of the first insertion hole 21. The front ends of the first retracting member 4A and knot pusher 5 are positioned near the front end of the body 2A. The second retracting member 4B is disposed in the second branching portion 21C of the first insertion hole 21. The front end of the second retracting member 4B is located near a branching position at which the extended portion 21A branches into the first branching portion 21B and second branching portion 21C.

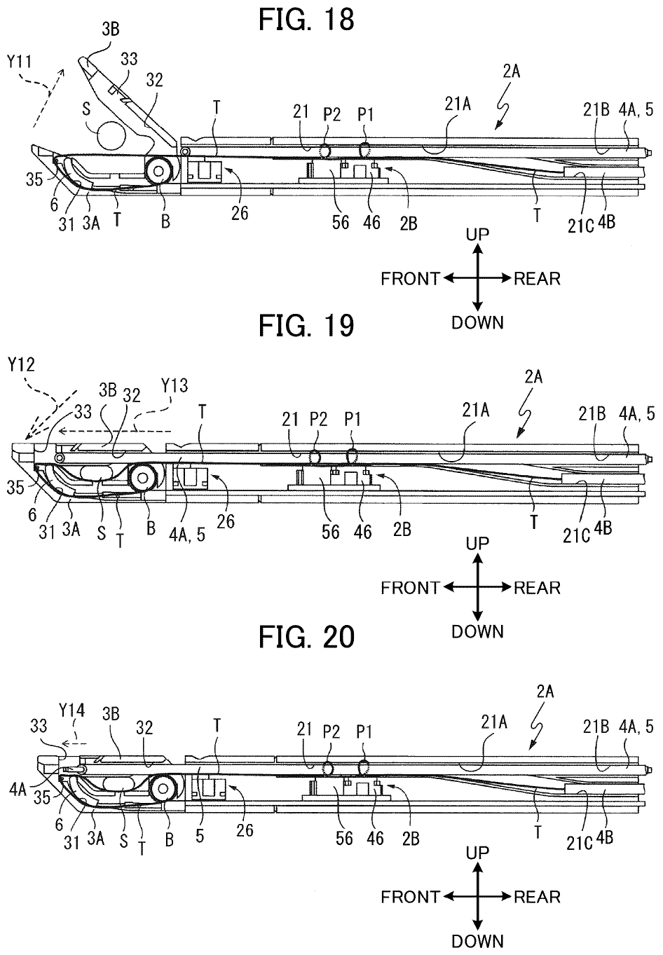

The thread T drawn off the bobbin B passes through the first insertion hole 31 of the first jaw part 3A, past the latching pin 35, and through the extended portion 21A and first branching portion 21B of the first insertion hole 21. A leading end of the thread Tis gripped by the hook body 43 and wire 44 of the second retracting member 4B. The first looping shaft 46 and second looping shaft 56 of the forming part 2B are disposed in the third rotational position. A first loop P1 is formed on the first looping shaft 46 and a second loop P2 is formed on the second looping shaft 56. The first retracting member 4A and the knot pusher 5 are inserted through the first loop P1 and second loop P2. To facilitate understanding, the first loop P1 and second loop P2 are depicted as being detached from the first looping shaft 46 and second looping shaft 56 in FIGS. 18 through 40.

The body 2A of the ligation device 1A is arranged in the living body at a position rearward of the ligation target S. In this state, the robot R drives the robot motors MR to operate the first control wire 201. As illustrated in FIG. 18, the second jaw part 3B moves from the adjacent position to the separated position (arrow Y11) in response to an operation on the first control wire 201. In this state, the body 2A is moved forward to arrange the ligation target S between the first jaw part 3A and second jaw part 3B.

Next, the robot R drives the robot motors MR to operate the first control wire 201. As illustrated in FIG. 19, the second jaw part 3B is moved from the separated position to the adjacent position (arrow Y12). The ligation target S is clamped between the first jaw part 3A and second jaw part 3B. The thread T is positioned beneath the ligation target S. Next, the ligation device 1A drives the first motor Ma1 and the second motor Mc1 to rotate the first threaded shaft Xa1 and the second threaded shaft Xc1 in the forward direction. The hook body 41 and wire 42 of the first retracting member 4A and the knot pusher 5 move forward (arrow Y13). The first retracting member 4A and the knot pusher 5 pass through the recess 32 in the second jaw part 3B. The front ends of the first retracting member 4A and knot pusher 5 reach the second insertion hole 33 in the second jaw part 3B.

Next, the ligation device 1A drives the first motor Ma1 to rotate the first threaded shaft Xa1 in the forward direction. As illustrated in FIG. 20, the hook body 41 and wire 42 of the first retracting member 4A move forward (arrow Y14). The front end of the first retracting member 4A arrives near the front edge of the second insertion hole 33 in the second jaw part 3B. Since the second motor Mc1 is not driven, the second threaded shaft Xc1 does not rotate, and the knot pusher 5 does not move forward. Therefore, the front end of the first retracting member 4A protrudes farther forward than the front end of the knot pusher 5.

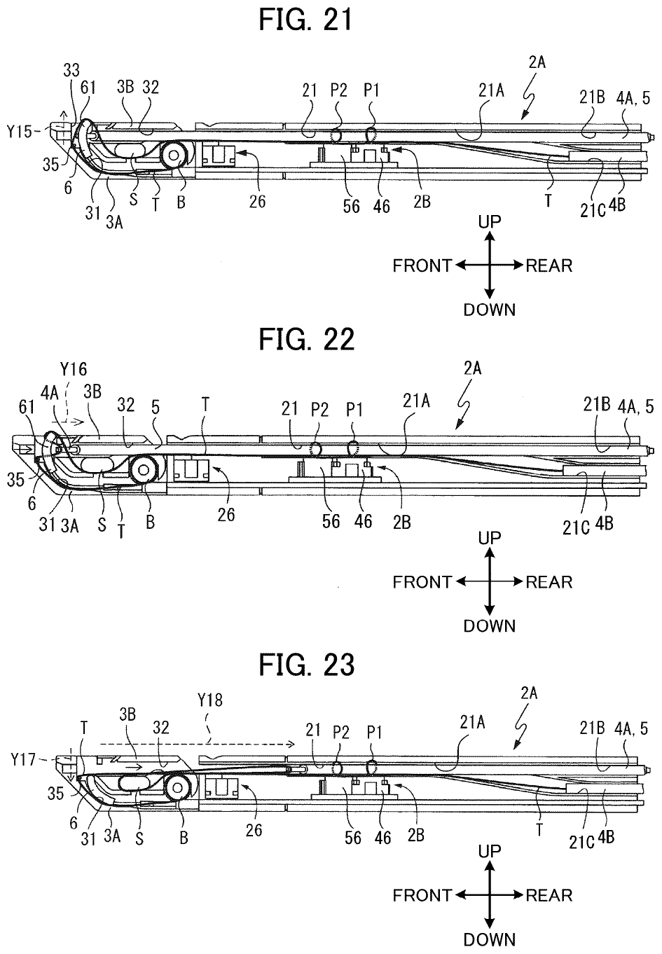

Next, the robot R drives the robot motors MR to operate the sixth control wire 251. The feeder 6 moves forward in response to an operation on the sixth control wire 251. As illustrated in FIG. 21, the base 61 of the feeder 6 protrudes upward from the first insertion hole 31 in the first jaw part 3A and moves farther upward through the second insertion hole 33 in the second jaw part 3B (arrow Y15). At this time, the groove 62 in the feeder 6 gripping the thread T raises the thread T upward.

Next, the ligation device 1A drives the first motor Ma1 and the second motor Mc1 to rotate the first threaded shaft Xa1 and the second threaded shaft Xc1 in the reverse direction. As illustrated in FIG. 22, the hook body 41 and wire 42 of the first retracting member 4A and the knot pusher 5 move rearward (arrow Y16). The front ends of the first retracting member 4A and knot pusher 5 move rearward and separate from the feeder 6. The thread T is hooked by the hook 41B of the hook body 41. Next, the ligation device 1A drives the first auxiliary motor Ma2 to rotate the first auxiliary shaft Xa2 in the forward direction. The wire 42 moves forward in response to the forward rotation of the first auxiliary shaft Xa2. The thread T becomes clamped between the front surface 42A of the wire 42 and the hook 41B of the hook body 41. In this state, the thread T is not pinched firmly and can move relative to the hook 41B of the hook body 41 and the wire 42. The hook 41B of the hook body 41 and the wire 42 grip the thread T with sufficient strength to restrict the thread T from coming off the hook body 41. Hereinafter, this gripping state will be called a “semi-gripping state.”

Next, the robot R drives the robot motors MR to operate the sixth control wire 251. As illustrated in FIG. 23, the feeder 6 moves rearward to be accommodated in the first insertion hole 31 of the first jaw part 3A (arrow Y17). The ligation device 1A also drives the first motor Ma1 and the second motor Mc1 to rotate the first threaded shaft Xa1 and the second threaded shaft Xc1 in the reverse direction. The hook body 41 and wire 42 of the first retracting member 4A together with the knot pusher 5 move rearward (arrow Y18). The thread T gripped by the first retracting member 4A passes over the ligation target S. The front ends of the first retracting member 4A and knot pusher 5 reach an area within the extended portion 21A of the first insertion hole 21 between the first cutter 26 and the forming part 2B.

Next, the ligation device 1A drives the first auxiliary motor Ma2 to rotate the first auxiliary shaft Xa2 in the forward direction. The wire 42 moves forward relative to the hook body 41. The thread T, which is gripped by the hook 41B of the hook body 41 and the wire 42, is pinched and becomes immovable relative to the hook 41B of the hook body 41 and the wire 42. Hereinafter, this gripping state will be called the “fully gripping state.”

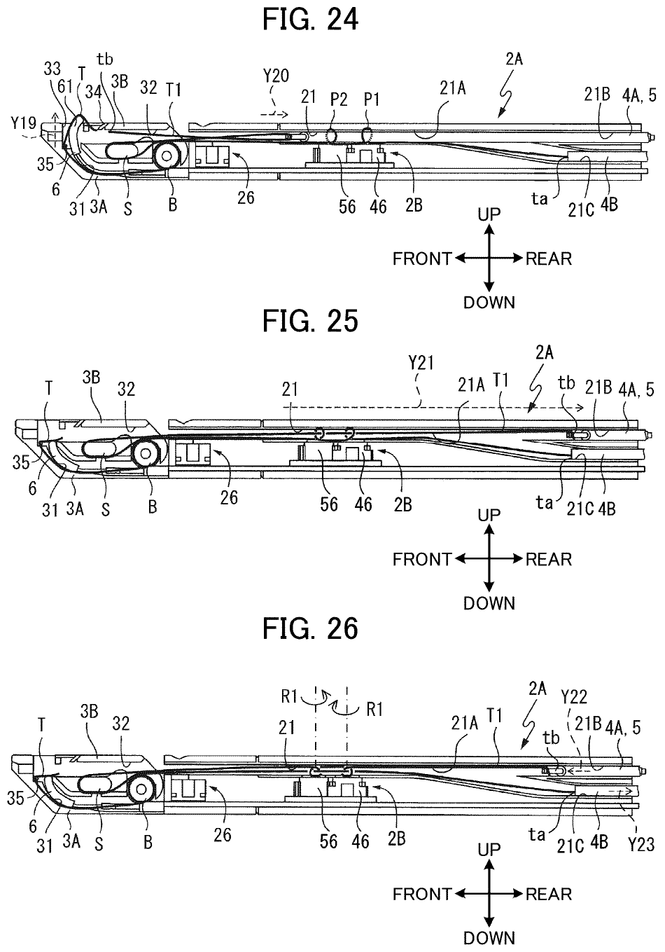

Next, the robot R drives the robot motors MR to operate the sixth control wire 251. The feeder 6 moves forward. As illustrated in FIG. 24, the base 61 of the feeder 6 protrudes upward from the first insertion hole 31 in the first jaw part 3A and moves farther upward through the second insertion hole 33 in the second jaw part 3B (arrow Y19). At this time, the groove 62 of the feeder 6 grips the thread T and lifts the thread T upward. Next, the ligation device 1A drives the first motor Ma1 and the second motor Mc1 to rotate the first threaded shaft Xa1 and the second threaded shaft Xc1 in the reverse direction. The hook body 41 and wire 42 of the first retracting member 4A along with the knot pusher 5 move rearward (arrow Y20).

The second cutter 34 fixed to the second jaw part 3B presses against a portion of the thread T between a portion gripped by the groove 62 of the feeder 6 and a portion gripped by the first retracting member 4A. The second cutter 34 cuts the thread T. By this cutting, a thread T1 (a portion of the thread T) is separated from a remaining portion of the thread T on the bobbin B side. The thread T1 has one end (first end ta) gripped by the second retracting member 4B, and an other end (second end tb). The thread T1 extends forward from the first end ta, wraps around the ligation target S, extends rearward, bends at the portion gripped by the first retracting member 4A, and extends forward to the second end tb. The thread T1 is wrapped around the ligation target S.

Next, the robot R drives the robot motors MR to operate the sixth control wire 251. As illustrated in FIG. 25, the feeder 6 moves rearward to be accommodated in the first insertion hole 31 of the first jaw part 3A. The ligation device 1A also drives the first auxiliary motor Ma2 to rotate the first auxiliary shaft Xa2 in the reverse direction. The wire 42 moves rearward relative to the hook body 41. The hook body 41 thus switches from the fully gripping state to the semi-gripping state.

The ligation device 1A drives the first motor Ma1 and the second motor Mc1 to rotate the first threaded shaft Xa1 and the second threaded shaft Xc1 in the reverse direction. The hook body 41 and wire 42 of the first retracting member 4A together with the knot pusher 5 move rearward (arrow Y21). As the first retracting member 4A and the knot pusher 5 move rearward, the second end tb of the thread T1 passes rearward through the first loop P1 and the second loop P2. The front ends of the first retracting member 4A and the knot pusher 5 arrive in the first branching portion 21B of the first insertion hole 21. The ligation device 1A also drives the first auxiliary motor Ma2 to rotate the first auxiliary shaft Xa2 in the forward direction. The wire 42 moves forward relative to the hook body 41. The hook body 41 switches from the semi-gripping state to the fully gripping state.

Next, the robot R drives the robot motors MR to operate the third control wire 221. As illustrated in FIG. 26, the first looping shaft 46 and the second looping shaft 56 rotate 360 degrees into the first rotating direction R1 from the third rotational position. The first loop P1 detaches from the first looping shaft 46 and the second loop P2 detaches from the second looping shaft 56.

The ligation device 1A also drives the first motor Ma1 and the second motor Mc1 to rotate the first threaded shaft Xa1 and the second threaded shaft Xc1 in the forward direction. The hook body 41 and wire 42 of the first retracting member 4A together with the knot pusher 5 move forward (arrow Y22). The ligation device 1A simultaneously drives the first motor Mb1 to rotate the first threaded shaft Xb1 in the reverse direction. The hook body 43 and wire 44 of the second retracting member 4B move rearward (arrow Y23). This restricts the thread T1 from slackening.

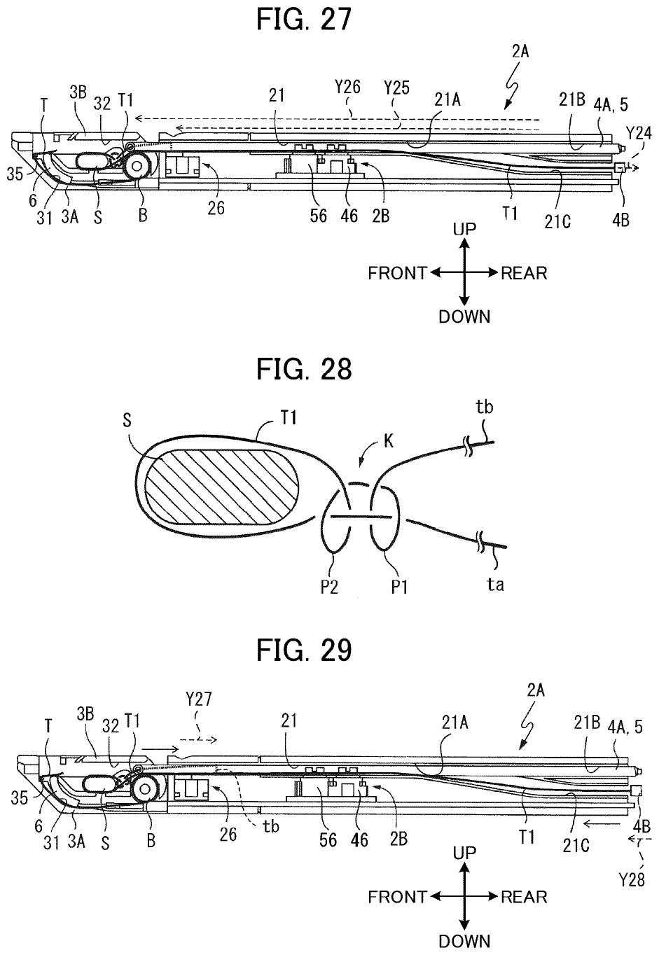

Next, the ligation device 1A drives the first motor Mb1 to rotate the first threaded shaft Xb1 in the reverse direction. As illustrated in FIG. 27, the hook body 43 and wire 44 of the second retracting member 4B move farther rearward (arrow Y24) in response to the rotation of the first threaded shaft Xb1. This applies tension to the thread T1. Next, the ligation device 1A drives the first motor Ma1 to rotate the first threaded shaft Xa1 in the forward direction. The hook body 41 and wire 42 of the first retracting member 4A move forward (arrow Y25). The front end of the first retracting member 4A arrives near the front end of the body 2A. The ligation device 1A also drives the second motor Mc1 to rotate the second threaded shaft Xc1 in the forward direction. The knot pusher 5 moves forward (arrow Y26). The front end of the knot pusher 5 protrudes forward from the front end of the body 2A and arrives near the ligation target S. As a result, the knot pusher 5 moves the first loop P1 and second loop P2 near the ligation target S. FIG. 28 illustrates the first loop P1 and the second loop P2 positioned near the ligation target S.

Next, the ligation device 1A drives the first motor Ma1 to rotate the first threaded shaft Xa1 in the reverse direction. As illustrated in FIG. 29, the hook body 41 and wire 42 of the first retracting member 4A move rearward (arrow Y27). The front end of the first retracting member 4A reaches a point in the first insertion hole 21 rearward of the first cutter 26 within the body 2A. The ligation device 1A also drives the first motor Mb1 to rotate the first threaded shaft Xb1 in the forward direction. The hook body 43 and wire 44 of the second retracting member 4B move forward (arrow Y28).

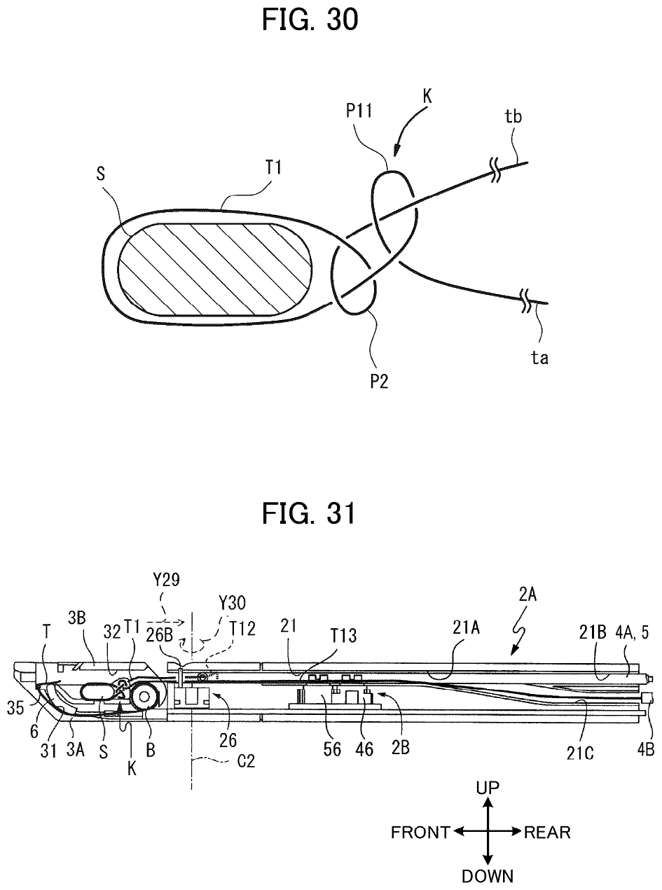

The movement of the first retracting member 4A and second retracting member 4B elongates the first loop P1 illustrated in FIG. 28. As illustrated in FIG. 30, a new first loop P11 is formed in a portion of the thread T1 between the second loop P2 and the first end ta. This operation forms a square knot K for ligating the ligation target S with the thread T1.

Next, the ligation device 1A drives the second motor Mc1 to rotate the second threaded shaft Xc1 in the reverse direction. As illustrated in FIG. 31, the knot pusher 5 moves rearward (arrow Y29). The front end of the knot pusher 5 arrives near the front end of the first retracting member 4A. Next, the robot R drives the robot motors MR to operate the fourth control wire 231 and the fifth control wire 232. The rotary base 26A of the first cutter 26 is rotated (arrow Y30). The blade 26B of the first cutter 26 separates, from the thread T1, a portion extending from the knot K toward the first end ta and another portion extending from the knot K toward the second end tb. Hereinafter, the portion of the thread T1 separated from the knot K that includes the second end tb will be called a “thread T12.” The portion of the thread T1 separated from the knot K that includes the first end ta will be called a “thread T13.”

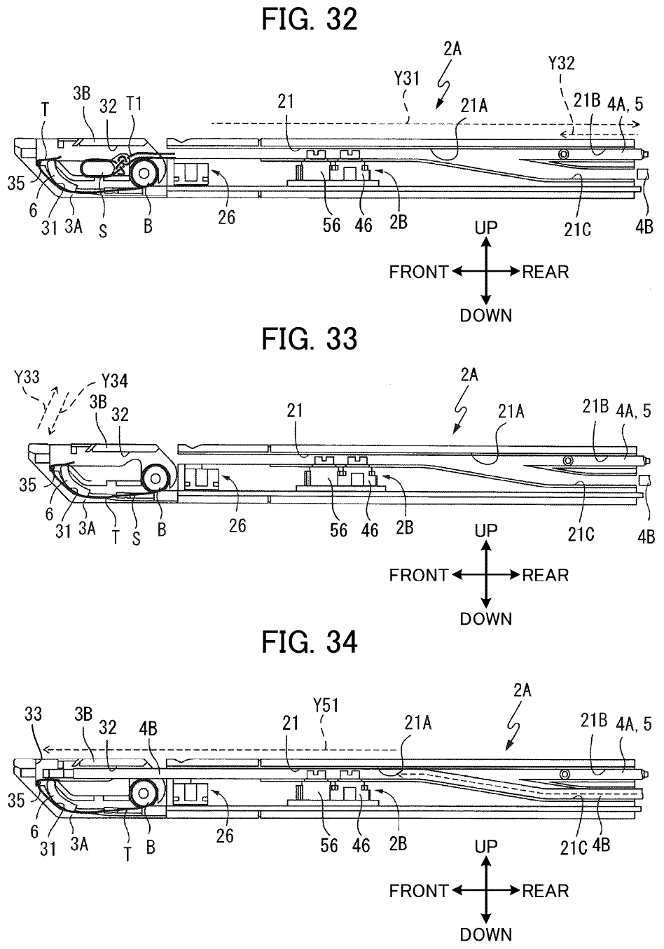

Next, the ligation device 1A drives the first motor Ma1 and the second motor Mc1 to rotate the first threaded shaft Xa1 and the second threaded shaft Xc1 in the reverse direction. As illustrated in FIG. 32, the hook body 41 and wire 42 of the first retracting member 4A along with the knot pusher 5 move rearward (arrow Y31). The front ends of the first retracting member 4A and the knot pusher 5 move to the rear of the body 2A. The thread T12 gripped by the first retracting member 4A (see FIG. 31) is removed from the first retracting member 4A. The thread T13 gripped by the second retracting member 4B (see FIG. 31) is removed from the second retracting member 4B. The ligation device 1A drives the first motor Ma1 to rotate the first threaded shaft Xa1 in the forward direction. The hook body 41 and wire 42 of the first retracting member 4A move forward (arrow Y32). The front end of the first retracting member 4A arrives in the first branching portion 21B of the first insertion hole 21.

Next, the robot R drives the robot motors MR to operate the first control wire 201. As illustrated in FIG. 33, the second jaw part 3B moves from the adjacent position to the separated position (arrow Y33). The ligation target S that has been ligated by the thread T1 (see FIG. 32) is removed from the holder 3. Next, the robot R drives the robot motors MR to operate the first control wire 201. The second jaw part 3B moves from the separated position to the adjacent position (arrow Y34).

### <Operations in a Repositioning Process>

A repositioning process will be described. This process returns the ligation device 1A to its initial state after the ligation target S was ligated with the thread T in the ligating process. First, the robot R drives the robot motors MR to operate the third control wire 221. The first looping shaft 46 and second looping shaft 56 are rotated 180 degrees from the third rotational position to the first rotational position.

The ligation device 1A drives the first motor Mb1 to rotate the first threaded shaft Xb1 in the forward direction. As illustrated in FIG. 34, the hook body 43 and wire 44 of the second retracting member 4B move forward (arrow Y51). The second retracting member 4B passes through the second branching portion 21C and extended portion 21A of the first insertion hole 21. The front end of the second retracting member 4B arrives near the front edge of the second insertion hole 33 in the second jaw part 3B.

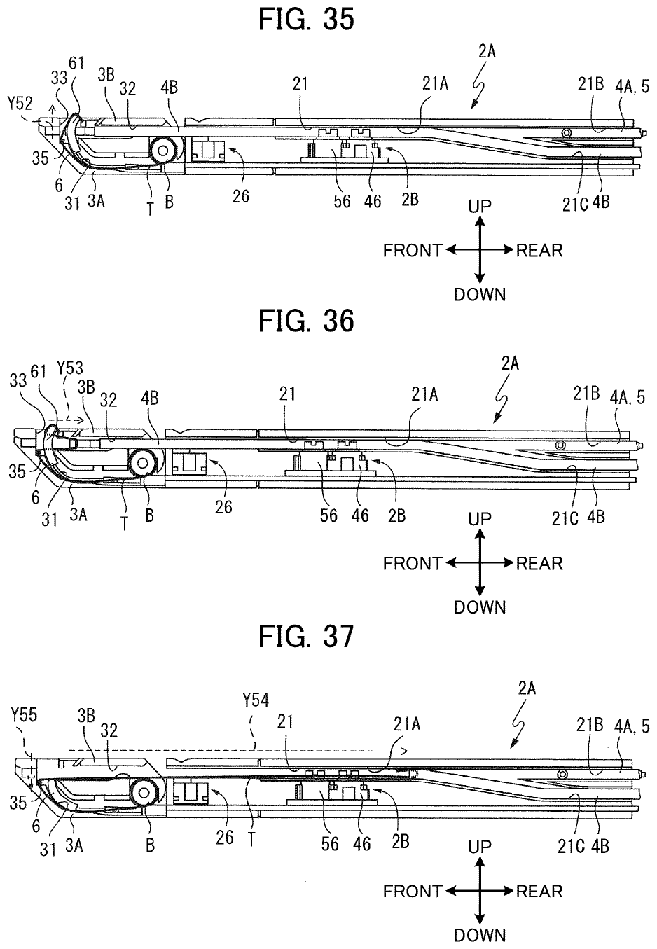

Next, the robot R drives the robot motors MR to operate the sixth control wire 251. The feeder 6 moves forward. As illustrated in FIG. 35, the base 61 of the feeder 6 protrudes upward from the first insertion hole 31 in the first jaw part 3A and moves farther upward through the second insertion hole 33 in the second jaw part 3B (arrow Y52). At this time, the groove 62 of the feeder 6 that grips the thread T raises the thread T upward.

Next, the ligation device 1A drives the first motor Mb1 to rotate the first threaded shaft Xb1 in the reverse direction. As illustrated in FIG. 36, the hook body 43 and wire 44 of the second retracting member 4B move rearward (arrow Y53). The thread T is thus hooked by the hook 43B of the hook body 43. Next, the ligation device 1A drives the first auxiliary motor Mb2 to rotate the first auxiliary shaft Xb2 in the forward direction. The wire 44 moves forward. The thread T is gripped between the front surface 44A of the wire 44 and the hook 43B of the hook body 43.

Next, the ligation device 1A drives the first motor Mb1 to rotate the first threaded shaft Xb1 in the reverse direction. As illustrated in FIG. 37, the hook body 43 and wire 44 of the second retracting member 4B move rearward (arrow Y54). The front end of the second retracting member 4B arrives near the branching position at which the extended portion 21A of the first insertion hole 21 branches into the first branching portion 21B and the second branching portion 21C. As a result, the thread T drawn off the bobbin B passes through the first insertion hole 31 of the first jaw part 3A, past the latching pin 35, and through the recess 32 of the second jaw part 3B and the extended portion 21A of the first insertion hole 21. A portion of the thread T is arranged in the forming part 2B along the second groove 460A of the first looping shaft 46 in the first rotational position and the third groove 560A of the second looping shaft 56 in the first rotational position (see FIG. 5). The robot R also drives the robot motors MR to operate the sixth control wire 251. The feeder 6 moves rearward to be accommodated in the first insertion hole 31 of the first jaw part 3A (arrow Y55).

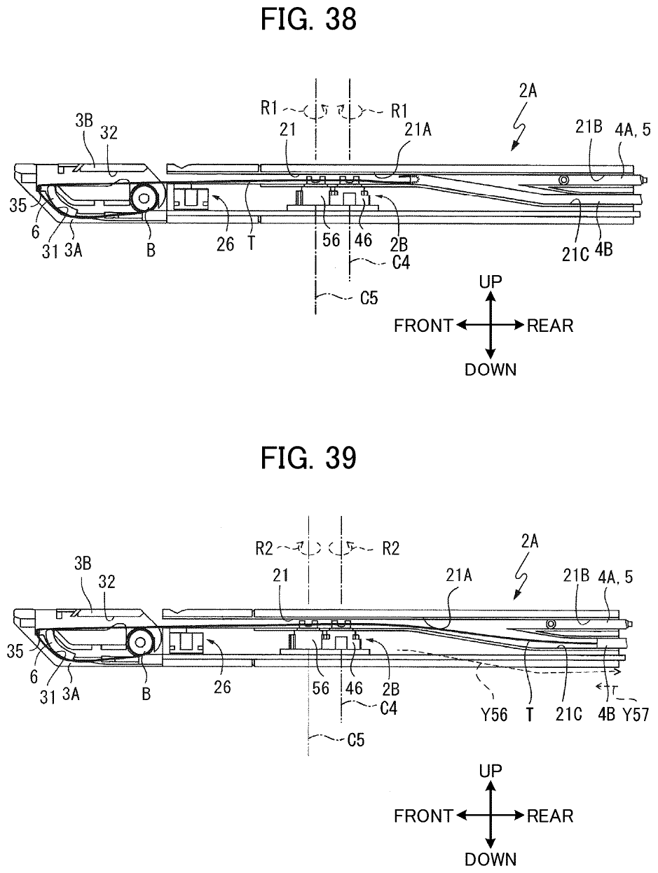

Next, the robot R drives the robot motors MR to operate the third control wire 221. As illustrated in FIG. 38, the first looping shaft 46 and the second looping shaft 56 are rotated 90 degrees in the first rotating direction R1 from the first rotational position to the second rotational position. This operation moves the thread T arranged along the second groove 460A into the fourth groove 460B and fifth groove 460C, and moves the thread T arranged along the third groove 560A into the sixth groove 560B and seventh groove 560C.

Next, the ligation device 1A drives the first motor Mb1 to rotate the first threaded shaft Xb1 in the reverse direction. As illustrated in FIG. 39, the second retracting member 4B moves rearward while gripping the thread T (arrow Y56). The front end of the second retracting member 4B arrives in the second branching portion 21C of the first insertion hole 21. Next, the ligation device 1A drives the first motor Mb1 to rotate the first threaded shaft Xb1 in the forward direction. The second retracting member 4B moves slightly forward (arrow Y57). The robot R also drives the robot motors MR to operate the third control wire 221. The first looping shaft 46 and the second looping shaft 56 rotate 270 degrees in the second rotating direction R2 from the second rotational position to the third rotational position. As a result, the first loop P1 is formed on the first looping shaft 46 and the second loop P2 is formed on the second looping shaft 56, as illustrated in FIG. 40.

Next, the ligation device 1A drives the second motor Mc1 to rotate the second threaded shaft Xc1 in the forward direction. The knot pusher 5 moves forward. The front end of the knot pusher 5 protrudes further forward than the first retracting member 4A. Next, the ligation device 1A drives the first motor Ma1 and the second motor Mc1 to rotate the first threaded shaft Xa1 and the second threaded shaft Xc1 in the forward direction. The hook body 41 and wire 42 of the first retracting member 4A together with the knot pusher 5 move forward (arrow Y58). The first retracting member 4A and knot pusher 5 pass through the first loop P1 formed on the first looping shaft 46 of the forming part 2B and the second loop P2 formed on the second looping shaft 56.

Additionally, each time the front end of the knot pusher 5 passes through the thread T forming the first loop P1 and the second loop P2, the ligation device 1A drives the second auxiliary motor Mc2 to rotate the second auxiliary shaft Xc2 alternately in the forward direction and the reverse direction. The knot pusher 5 rotates 180 degrees alternately toward one side and the other side about the seventh axis C7 (see FIG. 16; arrows Y59). In this way, the knot pusher 5 moves forward without catching on the first loop P1 and second loop P2. After passing through the first loop P1 and second loop P2, the front ends of the first retracting member 4A and the knot pusher 5 arrive near the front end of the body 2A. Through this process, the ligation device 1A is returned to its initial state.

### <Functions and Technical Advantages of the Ligation Device 1A>

The ligation device 1A is configured to ligate the ligation target S by using the retractor 4 to draw the thread T for ligating the ligation target S into the body 2A. By driving the hook body 41 of the first retracting member 4A with the first motor Ma1 and the hook body 43 of the second retracting member 4B with the first motor Mb1, the ligation device 1A can perform a stable operation to pull the thread T for ligating the ligation target S into the body 2A. Hence, the ligation device 1A can stably ligate the ligation target S with the thread T.

The ligation device 1A is configured to rotate the first threaded shaft Xa1 to move the first nut portion 710 meshed with the first threaded shaft Xa1 in the front-rear direction, whereby the hook body 41 connected to the first hook support part 70A is moved in the front-rear direction. The ligation device 1A is configured to rotate the first threaded shaft Xb1 to move the first nut portion 720 meshed with the first threaded shaft Xb1 in the front-rear direction, whereby the hook body 43 connected to the second hook support part 70B is moved in the front-rear direction. Therefore, the ligation device 1A can efficiently transmit the rotational drive forces of the first motors Ma1 and Mb1, which rotate the first threaded shafts Xa1 and Xb1, to the first hook support part 70A and second hook support part 70B, respectively, to move the hook bodies 41 and 43 in the front-rear direction.

The moving mechanism 71 is configured to move the wire 42 of the first retracting member 4A relative to the hook body 41 in the front-rear direction. The moving mechanism 72 moves the wire 44 of the second retracting member 4B relative to the hook body 43 in the front-rear direction. By stably gripping and drawing the thread T into the body 2A with the hook bodies 41 and 43 and the wires 42 and 44, the ligation device 1A can stably ligate the ligation target S with the thread T.

The moving mechanism 71 is disposed in the first drive mechanism 7A, and the moving mechanism 72 is disposed in the second drive mechanism 7B. Accordingly, the ligation device 1A can be made more compact in size than if the moving mechanism 71 were disposed separately from the first drive mechanism 7A and the moving mechanism 72 were disposed separately from the second drive mechanism 7B.

By driving the first auxiliary motor Ma2 to rotate the first auxiliary shaft Xa2, the moving mechanism 71 is configured to move the wire 42 in the front-rear direction via the first main gear 71B, first follow gear 71C, and first converter 71D. By driving the first auxiliary motor Mb2 to rotate the first auxiliary shaft Xb2, the moving mechanism 72 is configured to move the wire 44 in the front-rear direction via the first main gear 72B, first follow gear 72C, and first converter 72D. Thus, the ligation device 1A can stably move the hook bodies 41 and 43 and the wires 42 and 44 in the front-rear direction by having first motors Ma1 and Mb1 for moving the hook bodies 41 and 43 separate from first auxiliary motors Ma2 and Mb2 for moving the wires 42 and 44.

The first hook support part 70A and the second hook support part 70B are at mutually different positions in directions orthogonal to the front-rear direction (i.e., in the up-down and left-right directions). When viewed from the rear, the first hook support part 70A and second hook support part 70B do not overlap each other. The first hook support part 70A and second hook support part 70B do not interfere with each other even when placed close together in the front-rear direction. Hence, the first hook support part 70A can move further forward than the second hook support part 70B, and the second hook support part 70B can move further forward than the first hook support part 70A. This structure enables the ligation device 1A to move each of the first hook support part 70A and second hook support part 70B over a greater range in the front-rear direction than otherwise.

The ligation device 1A is configured to rotate the second threaded shaft Xc1 to move the second nut portion 730 meshed with the second threaded shaft Xc1 in the front-rear direction, whereby the knot pusher 5 connected to the pusher support part 70C moves in the front-rear direction. Therefore, the ligation device 1A can efficiently transmit the rotational drive force of the second motor Mc1, which rotates the second threaded shaft Xc1, to the pusher support part 70C to move the knot pusher 5 in the front-rear direction.

The rotating mechanism 73 rotates the knot pusher 5 about the seventh axis C7. Since the rotating mechanism 73 is disposed in the third drive mechanism 7C, the ligation device 1A can be made more compact in size than if the rotating mechanism 73 were disposed separately from the third drive mechanism 7C. Further, by driving the second auxiliary motor Mc2 to rotate the second auxiliary shaft Xc2, the rotating mechanism 73 rotates the knot pusher 5 via the second main gear 73B and the second follow gear 739. By having the second motor Mc1 for moving the knot pusher 5 in the front-rear direction separate from the second auxiliary motor Mc2 for rotating the knot pusher 5, the ligation device 1A can stably move and rotate the knot pusher 5.

The first retracting member 4A and the knot pusher 5 are arranged concentrically. The part of the first hook support part 70A to which the first retracting member 4A is connected and the part of the pusher support part 70C to which the knot pusher 5 is connected overlap each other in the front-rear direction (when viewed in the front-rear direction). In this case, the first hook support part 70A and pusher support part 70C in the ligation device 1A can be arranged close together in directions orthogonal to the front-rear direction (i.e., in the up-down and left-right directions). Thus, the size of the ligation device 1A can be reduced in the directions orthogonal to the front-rear direction.

The distance L13 in the front-rear direction between the body 2A and the pusher support part 70C is shorter than the distance L11 in the front-rear direction between the body 2A and the first hook support part 70A. In this case, the ligation device 1A can restrict the pusher support part 70C from interfering with the first hook support part 70A when the knot pusher 5 is moved forward. Hence, the ligation device 1A can impede the first hook support part 70A from restricting movement of the pusher support part 70C.

The robot R connected to the robot connector 9 has the built-in robot motors MR for driving the second jaw part 3B, the feeder 6, and the forming part 2B. That is, the ligation device 1A does not have motors for driving the second jaw part 3B, the feeder 6, and the forming part 2B. Accordingly, the ligation device 1A can be made smaller and lighter.

The drive motors Mm are arranged closer to the robot connector 9 than the threaded shafts X1 and auxiliary shafts X2 are to the robot connector 9. The drive motors Mm are also closer to the robot connector 9 than the first hook support part 70A, second hook support part 70B, and pusher support part 70C are to the robot connector 9. By arranging the drive motors Mm at a position close to the robot connector 9 in this way, the force acting on the robot connector 9 while the robot R holds the ligation device 1A is smaller than if the drive motors Mm were arranged at a position separated from the robot connector 9. This configuration allows the robot R to hold the ligation device 1A securely.

### Second Embodiment (Ligation Device 1B)

A ligation device 1B according to a second embodiment as one of the embodiments of the present disclosure will be described with reference to the drawings.

The ligation device 1B includes the body 2A, forming part 2B, holder 3, retractor 4 (first retracting member 4A and second retracting member 4B), knot pusher 5, feeder 6, and robot connector 9 having the same configuration as those in the ligation device 1A (see FIG. 1). The ligation device 1B differs from the ligation device 1A in that the ligation device 1B includes a drive unit 8 instead of the drive unit 7. The configuration of the drive unit 8, which differs from that of the drive unit 7, will be described below, while descriptions of other configurations are omitted. The drive motors Mm in the drive unit 7 illustrated in FIG. 1 include the first motor Ma1, first auxiliary motor Ma2, first motor Mb1, first auxiliary motor Mb2, second motor Mc1, and second auxiliary motor Mc2 (see FIG. 6). However, the drive unit 8 includes only the first motor Ma1, first motor Mb1, and second motor Mc1 and does not include the first auxiliary motor Ma2, first auxiliary motor Mb2, and second auxiliary motor Mc2.

### <Drive Unit 8>

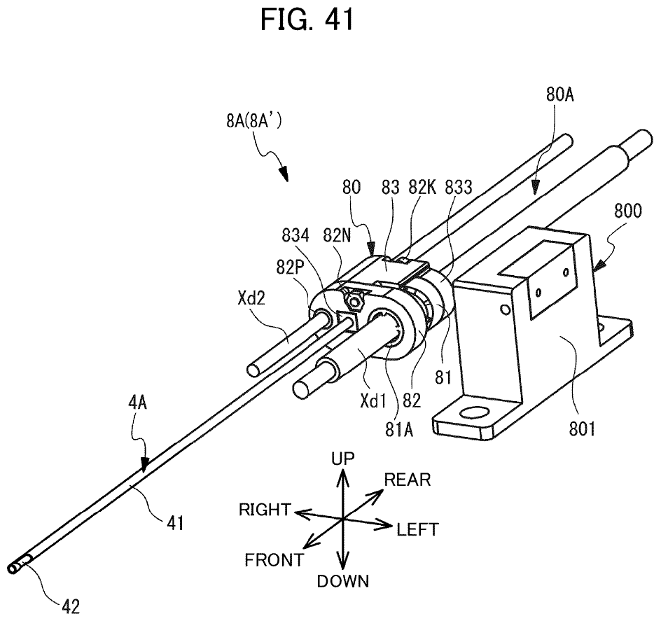

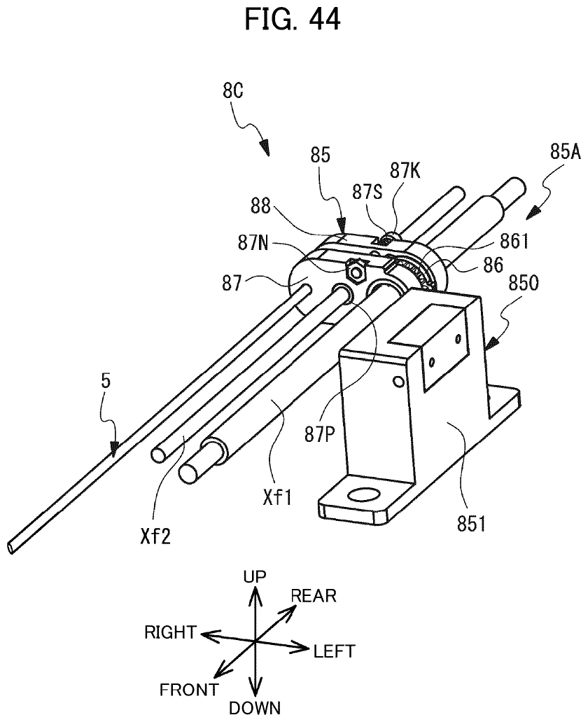

The drive unit 8 includes a first drive mechanism 8A (see FIGS. 41-43), a second drive mechanism 8A′, and a third drive mechanism 8C (see FIGS. 44-46). The first drive mechanism 8A is configured to move the hook body 41 and wire 42 of the first retracting member 4A in the front-rear direction. The second drive mechanism 8A′ is configured to move the hook body 43 and wire 44 of the second retracting member 4B in the front-rear direction. The second drive mechanism 8A′ has the same configuration as the first drive mechanism 8A. A description of the configuration and operations of the second drive mechanism 8A′ is omitted below. The third drive mechanism 8C is configured to move the knot pusher 5 in the front-rear direction and rotate the same.

### <First Drive Mechanism 8A>

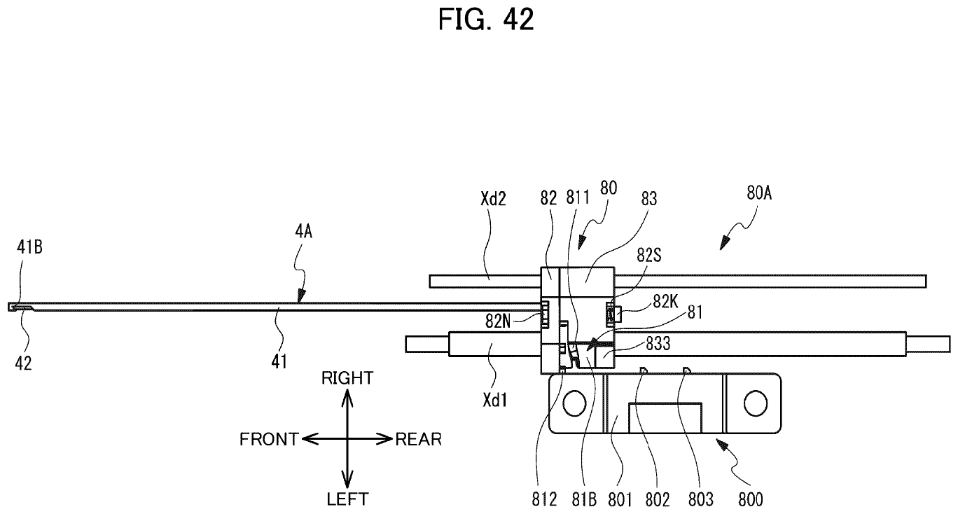

As illustrated in FIGS. 41 and 42, the first drive mechanism 8A includes the first motor Ma1 (see FIG. 6), a first threaded shaft Xd1, a support shaft Xd2, a first hook support part 80, and a first switching unit 800.

The first threaded shaft Xd1 and the support shaft Xd2 extend in the front-rear direction. The first threaded shaft Xd1 and support shaft Xd2 are parallel to each other. The first threaded shaft Xd1 is rotatably supported by the second support plate 702 and the base plate 705 (see FIG. 6). A male thread is formed on a side surface of the first threaded shaft Xd1. The male thread is spiral shaped and extends in the front-rear direction. A gear is connected to a portion of the first threaded shaft Xd1 that protrudes forward from the second support plate 702 and meshes with a gear connected to the rotational shaft of the first motor Ma1. The gears have been omitted from FIGS. 41 and 42. The first threaded shaft Xd1 is configured to rotate when the first motor Ma1 is driven. The support shaft Xd2 has a columnar shape. The support shaft Xd2 is fixed between the second support plate 702 and the base plate 705 and is non-rotatable.

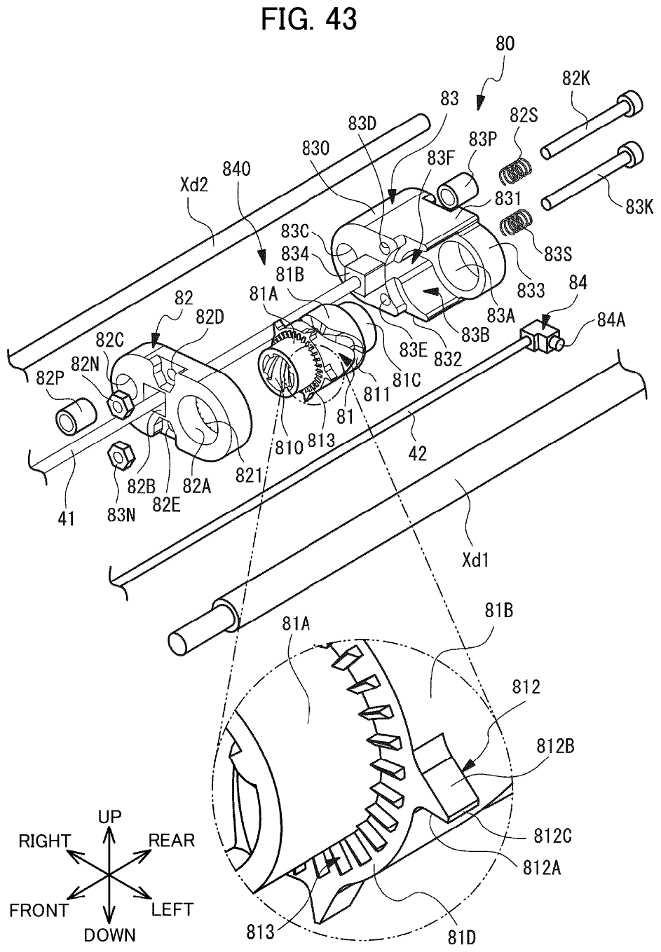

The first hook support part 80 supports the hook body 41 and wire 42 of the first retracting member 4A. As illustrated in FIG. 43, the first hook support part 80 has a first rotary body 81, first rotary body support parts 82 and 83, and a wire connector 84.

The first rotary body 81 has a hollow cylindrical shape. The first rotary body 81 defines a central axis that extends in the front-rear direction. The first rotary body 81 has a through-hole whose diameter is constant over the front-rear direction. A female thread is formed in an inner surface defining the through-hole. The female thread has a spiral shape that extends in the front-rear direction. The through-hole having this female thread formed in the inner surface will be called a “first nut portion 810.” The first threaded shaft Xd1 (see FIG. 41) is inserted in the first nut portion 810. The male thread on the first threaded shaft Xd1 meshes with the female thread in the first nut portion 810.

As illustrated in FIG. 43, the first rotary body 81 has a front portion 81A, a center portion 81B, and a rear portion 81C. The front portion 81A, center portion 81B, and rear portion 81C are arranged in line in the front-rear direction. The front portion 81A is positioned farthest forward, and the rear portion 81C is positioned farthest rearward. The center portion 81B is sandwiched in the front-rear direction by the front portion 81A and the rear portion 81C. The center portion 81B has a larger outer diameter than the front portion 81A and rear portion 81C. A stepped surface 81D is formed to connect respective side surfaces of the center portion 81B and front portion 81A to each other. The stepped surface 81D is orthogonal to the front-rear direction.

A groove 811 is formed in the side surface of the center portion 81B. The groove 811 extends meanderingly in a circumferential direction of the center portion 81B. A plurality of protrusions 812 is disposed on the side surface of the center portion 81B along a front edge thereof. The protrusions 812 protrude outward in radial directions with respect to the central axis of the first rotary body 81. Each protrusion 812 includes a first surface 812A, a second surface 812B, and an endface 812C. The endface 812C is the outermost portion of the first rotary body 81 in the radial direction with respect to the central axis of the same. When viewed from the front, the first surface 812A extends in a direction from the endface 812C toward the central axis of the first rotary body 81. When viewed from the front, the second surface 812B slopes in the clockwise direction relative to the direction from the endface 812C toward the central axis of the first rotary body 81.

A plurality of protrusions 813 is disposed on the stepped surface 81D. The protrusions 813 protrude frontward from the stepped surface 81D. The protrusions 813 are spaced at equal intervals in a circumferential direction about the central axis of the first rotary body 81.

The first rotary body support parts 82 and 83 are aligned in the front-rear direction. The first rotary body support part 82 is arranged on the front side of the first rotary body support part 83. Formed in the first rotary body support part 82 are a first through-hole 82A, a second through-hole 82B, a third through-hole 82C, a fourth through-hole 82D, and a fifth through-hole 82E. The first through-hole 82A, second through-hole 82B, third through-hole 82C, fourth through-hole 82D, and fifth through-hole 82E extend in the front-rear direction.

The first through-hole 82A has a circular cross-sectional shape. The front portion 81A of the first rotary body 81 is inserted in the first through-hole 82A from the rear. A plurality of recesses 821 is formed in a rear surface of the first rotary body support part 82 around the first through-hole 82A. The recesses 821 are spaced at equal intervals in a circumferential direction about the central axis of the first through-hole 82A.

The second through-hole 82B is positioned to the right of the first through-hole 82A. The second through-hole 82B has a square cross-sectional shape. The third through-hole 82C is positioned to the right of the second through-hole 82B. The third through-hole 82C has a circular cross-sectional shape. A first spacer 82P is disposed in the third through-hole 82C, and the support shaft Xd2 is inserted in the first spacer 82P. The fourth through-hole 82D is arranged above the second through-hole 82B. The fifth through-hole 82E is arranged below the second through-hole 82B. The fourth through-hole 82D and the fifth through-hole 82E both have circular cross-sectional shapes.

The first rotary body support part 83 has a base 830, a first extended part 831, a second extended part 832, a third extended part 833, and a protruding part 834. The base 830 has a right end that is curved into an arcuate shape. A first through-hole 83C is formed in the base 830. The first through-hole 83C extends in the front-rear direction. The first through-hole 83C has a circular cross-sectional shape. The first through-hole 83C is arranged rearward of the third through-hole 82C in the first rotary body support part 82. A second spacer 83P is disposed in the first through-hole 83C, and the support shaft Xd2 is inserted in the second spacer 83P.

The first extended part 831 extends leftward from near an upper end portion of the base 830. The second extended part 832 extends leftward from near a lower end portion of the base 830. The first extended part 831 and the second extended part 832 are spaced apart from each other in the up-down direction. A second through-hole 83D is formed in the first extended part 831. The second through-hole 83D is arranged rearward of the fourth through-hole 82D in the first rotary body support part 82. A third through-hole 83E is formed in the second extended part 832. The third through-hole 83E is arranged rearward of the fifth through-hole 82E in the first rotary body support part 82. The second through-hole 83D and the third through-hole 83E extend in the front-rear direction. The second through-hole 83D and the third through-hole 83E have circular cross-sectional shapes. The third extended part 833 extends leftward from near a rear end portion of the base 830.

A second space 83F is formed in a gap between the first extended part 831 and the second extended part 832 in a region near a right end of the gap. The front side of the second space 83F is covered by the protruding part 834. The protruding part 834 protrudes frontward from a front surface of the base 830. The protruding part 834 is arranged in the second through-hole 82B of the first rotary body support part 82. A through-hole extending in the front-rear direction is formed in the protruding part 834. The hook body 41 is connected to a front surface of the protruding part 834. The hook body 41 extends forward from the front surface of the protruding part 834. The through-hole in the hook body 41 and the through-hole in the protruding part 834 are arranged in line in the front-rear direction.

A first space 83B is formed in the gap between the first extended part 831 and the second extended part 832 in a region near a left end of the gap. The center portion 81B of the first rotary body 81 is arranged in the first space 83B. A through-hole 83A is formed in the third extended part 833 to the rear of the first space 83B. The through-hole 83A has a circular cross-sectional shape. The rear portion 81C of the first rotary body 81 is arranged in the through-hole 83A.

A first bolt 82K is inserted in the second through-hole 83D from the rear side of the first rotary body support part 83. The first bolt 82K passes through the second through-hole 83D in the first rotary body support part 83 and the fourth through-hole 82D in the first rotary body support part 82. A second bolt 83K is inserted in the third through-hole 83E from the rear side of the first rotary body support part 83. The second bolt 83K passes through the third through-hole 83E in the first rotary body support part 83 and the fifth through-hole 82E in the first rotary body support part 82. Front end portions of the first bolt 82K and the second bolt 83K protrude forward from the first rotary body support part 82. A first nut 82N is fitted onto the protruding portion (front end portion) of the first bolt 82K. A second nut 83N is fitted onto the protruding portion (front end portion) of the second bolt 83K.

A first spring 82S is interposed between the head of the first bolt 82K and a rear surface of the first rotary body support part 83. A second spring 83S is interposed between the head of the second bolt 83K and the rear surface of the first rotary body support part 83. The first spring 82S and the second spring 83S apply urging forces in a direction for moving the first rotary body support parts 82 and 83 toward each other. As a result, the rear surface of the first rotary body support part 82 and the front surface of the base 830 in the first rotary body support part 83 are in close contact with each other. The first rotary body 81 is sandwiched in the front-rear direction and supported by the first rotary body support part 82 and the third extended part 833 of the first rotary body support part 83. The protrusions 813 on the first rotary body 81 are fitted in the recesses 821 in the first rotary body support part 82 from the rear side.

As illustrated in FIG. 42, in a state where the first rotary body 81 is supported by the first rotary body support parts 82 and 83, a left edge of the first rotary body support part 82 is located at the same position in the left-right direction as the left edges of protrusions 812 on the first rotary body 81. The left edge of a portion of the center portion 81B excluding the protrusions 812 and the left edge of the third extended part 833 in the first rotary body support part 83 are positioned rightward relative to the left edge of the first rotary body support part 82.

As illustrated in FIG. 43, the wire connector 84 is connected to the rear end of the wire 42. The wire connector 84 is placed in the second space 83F of the first rotary body support part 83 and is movable in the front-rear direction. The wire 42 extending forward from the wire connector 84 passes through the through-hole in the protruding part 834 arranged frontward of the second space 83F, and further passes through the through-hole in the hook body 41 connected to the front surface of the protruding part 834.

The wire connector 84 has a left surface on which a cam follower 84A is disposed. The cam follower 84A is fitted in the groove 811 of the first rotary body 81. The wire 42 is coupled to the first rotary body 81 via the cam follower 84A and the groove 811.

As illustrated in FIGS. 41 and 42, the first switching unit 800 is arranged to the left of the first hook support part 80. As illustrated in FIG. 42, the first switching unit 800 includes a base 801, a first switching rod 802, and a second switching rod 803. The base 801 is fixed in the case 704 (see FIG. 1). A right surface of the base 801 and the left surface of the first rotary body support part 82 are at approximately the same position as each other in the left-right direction. The first switching rod 802 and second switching rod 803 extend in the left-right direction. The first switching rod 802 and second switching rod 803 are aligned in the front-rear direction. The first switching rod 802 is disposed frontward of the second switching rod 803. The first switching rod 802 and second switching rod 803 are arranged farther rearward than the first rotary body support part 82 of the first hook support part 80.

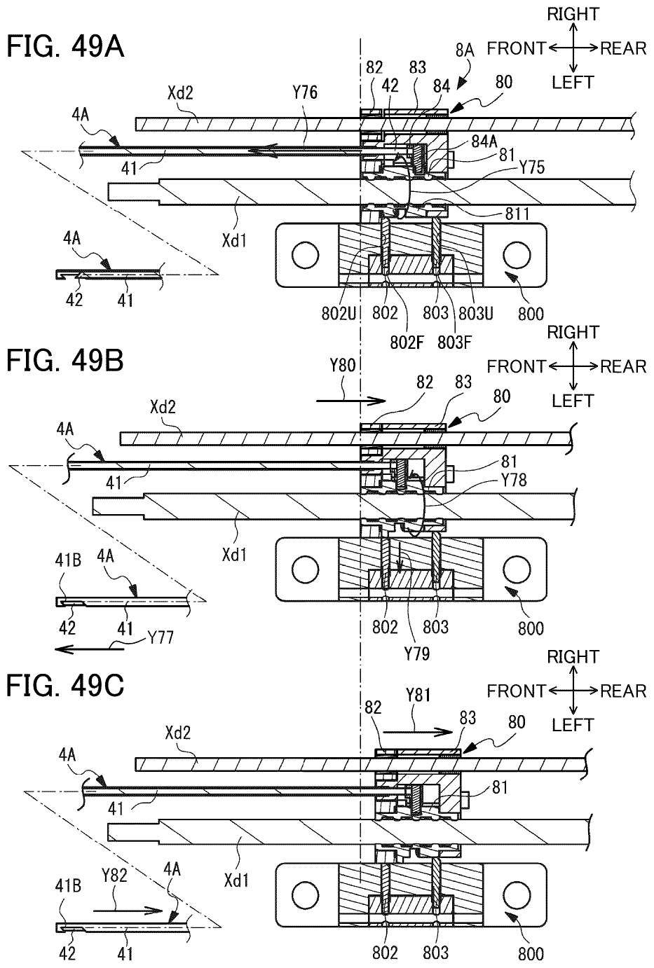

The first switching rod 802 is movable in the left-right direction along a first recess 802U (see FIG. 49A) formed in the right surface of the base 801. The second switching rod 803 is movable in the left-right direction along a second recess 803U (see FIG. 49A) formed in the right surface of the base 801. When the first switching rod 802 and second switching rod 803 are moved to their rightmost positions, distal ends of the first switching rod 802 and second switching rod 803 protrude rightward from the right surface of the base 801. When the first switching rod 802 and second switching rod 803 are moved to their leftmost positions, their distal ends do not protrude rightward from the right surface of the base 801. The first switching rod 802 is urged rightward by a first urging member 802F (see FIG. 49A) disposed at the bottom of the first recess 802U formed in the base 801. The second switching rod 803 is urged rightward by a second urging member 803F (see FIG. 49A) disposed at the bottom of the second recess 803U formed in the base 801.

### <Third Drive Mechanism 8C>

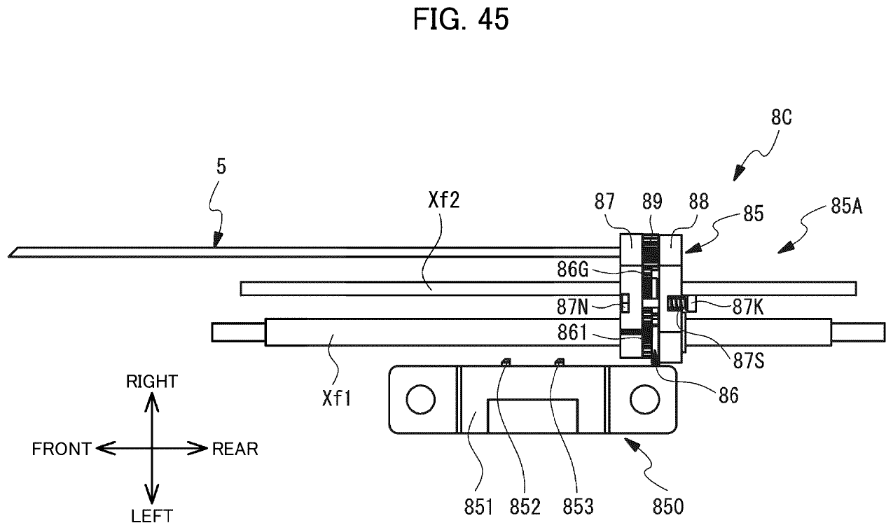

As illustrated in FIGS. 44 and 45, the third drive mechanism 8C includes the second motor Mc1 (see FIG. 6), a second threaded shaft Xf1, a support shaft Xf2, a pusher support part 85, and a second switching unit 850. The second threaded shaft Xf1, support shaft Xf2, pusher support part 85, and second switching unit 850 have common configurations with the first threaded shaft Xd1, support shaft Xd2, first hook support part 80, and first switching unit 800 of the first drive mechanism 8A (see FIGS. 41-43), with some exceptions. Below, descriptions of parts having common configurations will be omitted or simplified.

The second threaded shaft Xf1 and support shaft Xf2 correspond to the first threaded shaft Xd1 and support shaft Xd2 of the first drive mechanism 8A (see FIGS. 41-43), respectively. A gear is connected to a portion of the second threaded shaft Xf1 that protrudes frontward from the second support plate 702 (see FIG. 6) and meshes with a gear connected to the rotational shaft of the second motor Mc1. The gears have been omitted from FIGS. 44 and 45. The second threaded shaft Xf1 is configured to rotate when the second motor Mc1 is driven. The pusher support part 85 supports the knot pusher 5.

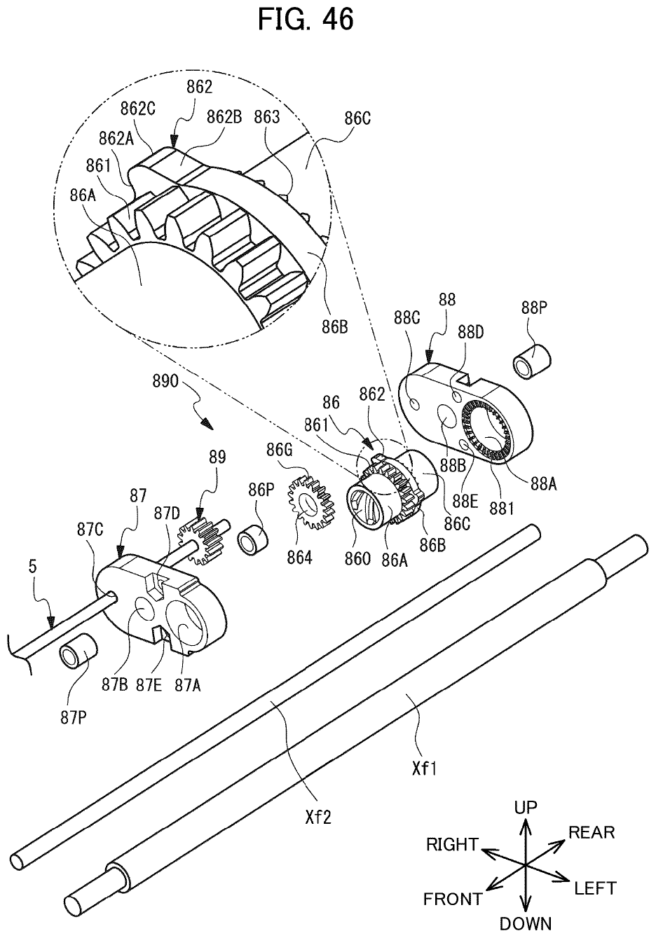

As illustrated in FIG. 46, the pusher support part 85 has a second rotary body 86, second rotary body support parts 87 and 88, and a first follow gear 89. The second rotary body 86 corresponds to the first rotary body 81 of the first drive mechanism 8A (see FIG. 43). A through-hole in the second rotary body 86 having a female thread formed in an inner surface thereof will be called a “second nut portion 860.” The second threaded shaft Xf1 is inserted in the second nut portion 860 (see FIG. 44). A male thread on the second threaded shaft Xf1 meshes with the female thread in the second nut portion 860. The second rotary body 86 has a front portion 86A, a center portion 86B, and a rear portion 86C that correspond to the front portion 81A, center portion 81B, and rear portion 81C of the first drive mechanism 8A (see FIG. 43), respectively.

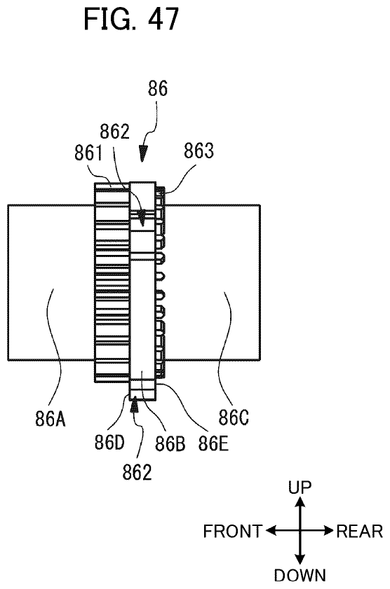

As illustrated in FIG. 47, a first stepped surface 86D is formed to connect respective side surfaces of the front portion 86A and the center portion 86B, and a second stepped surface 86E is formed to connect the respective side surfaces of the center portion 86B and the rear portion 86C. The first stepped surface 86D and second stepped surface 86E are orthogonal to the front-rear direction. A second main gear 861 is disposed on a rear end of the front portion 86A and frontward of the first stepped surface 86D. A plurality of protrusions 863 is disposed on the second stepped surface 86E. The protrusions 863 protrude rearward from the second stepped surface 86E. The protrusions 863 are arranged at equal intervals in a circumferential direction about the central axis of the second rotary body 86.

As illustrated in FIG. 46, a plurality of protrusions 862 is disposed on the side surface of the center portion 86B. The protrusions 862 protrude outward in radial directions with respect to the central axis of the second rotary body 86. Each protrusion 862 includes a first surface 862A, a second surface 862B, and an endface 862C. The endface 862C is the outermost portion of the second rotary body 86 in the radial direction with respect to the central axis of the same. When viewed from the front, the first surface 862A extends in a direction from the endface 862C toward the central axis of the second rotary body 86 and then extends further while curving in the counterclockwise direction. When viewed from the front, the second surface 862B slopes in the clockwise direction relative to the direction from the endface 862C toward the central axis of the second rotary body 86. Unlike the first rotary body 81 of the first drive mechanism 8A (see FIG. 43), no grooves are formed in the side surface of the center portion 86B.

The second rotary body support parts 87 and 88 correspond to the first rotary body support parts 82 and 83 of the first drive mechanism 8A (see FIG. 43), respectively. The second rotary body support part 87 has a first through-hole 87A, a second through-hole 87B, a third through-hole 87C, a fourth through-hole 87D, and a fifth through-hole 87E that correspond to the first through-hole 82A, second through-hole 82B, third through-hole 82C, fourth through-hole 82D, and fifth through-hole 82E of the first rotary body support part 82 (see FIG. 43), respectively. The front portion 86A of the second rotary body 86 is inserted in the first through-hole 87A from its rear side. A second spacer 87P is disposed in the second through-hole 87B, and the support shaft Xf2 is inserted in the second spacer 87P.

Formed in the second rotary body support part 88 are a first through-hole 88A, a second through-hole 88B, a third through-hole 88C, a fourth through-hole 88D, and a fifth through-hole 88E. The first through-hole 88A, second through-hole 88B, third through-hole 88C, fourth through-hole 88D, and fifth through-hole 88E extend in the front-rear direction. The first through-hole 88A has a circular cross-sectional shape. The rear portion 86C of the second rotary body 86 is inserted in the first through-hole 88A from its front side. A plurality of recesses 881 is formed in a front surface of the second rotary body support part 88 around the first through-hole 88A. The recesses 881 are spaced apart at equal intervals in a circumferential direction about an axis passing through the center of the first through-hole 88A.

The second through-hole 88B is disposed rightward of the first through-hole 88A. The second through-hole 88B has a circular cross-sectional shape. A third spacer 88P is disposed in the second through-hole 88B, and the support shaft Xf2 is inserted in the third spacer 88P. The third through-hole 88C is disposed rightward of the second through-hole 88B. The third through-hole 88C has a circular cross-sectional shape. The rear end of the knot pusher 5 is inserted in the third through-hole 88C. The fourth through-hole 88D is arranged above the second through-hole 88B. The fifth through-hole 88E is arranged below the second through-hole 88B. The fourth through-hole 88D and the fifth through-hole 88E have circular cross-sectional shapes.

As illustrated in FIGS. 44 and 45, a bolt 87K is inserted in the fourth through-hole 88D (see FIG. 46) from the rear side of the second rotary body support part 88. The bolt 87K passes through the fourth through-hole 88D in the second rotary body support part 88 and the fourth through-hole 87D in the second rotary body support part 87. A front end portion of the bolt 87K protrudes forward from the second rotary body support part 87. A nut 87N is fitted onto the protruding portion (front end portion) of the bolt 87K. A spring 87S is interposed between the head of the bolt 87K and the rear surface of the second rotary body support part 88. The spring 87S applies an urging force in a direction for moving the second rotary body support parts 87 and 88 toward each other. A bolt not illustrated in the drawings is inserted in the fifth through-holes 87E and 88E (see FIG. 46) from the rear of the second rotary body support part 88. A front end portion of the bolt protrudes forward from the second rotary body support part 87 and is fitted into a nut not illustrated in the drawings. A spring not illustrated in the drawings is interposed between the head of the bolt and the rear surface of the second rotary body support part 88. The spring applies an urging force in a direction for moving the second rotary body support parts 87 and 88 toward each other.

Through the urging forces of the spring 87S and the non-illustrated spring, the second rotary body 86 is sandwiched in the front-rear direction and supported by the second rotary body support parts 87 and 88. The protrusions 863 on the second rotary body 86 are fitted in the recesses 881 in the second rotary body support part 88 from its front side.

As illustrated in FIG. 46, a second gear 86G is arranged to the rear of the second rotary body support part 87, to the front of the second rotary body support part 88, and to the right of the second rotary body 86. The second gear 86G defines a rotational axis that extends in the front-rear direction. The second gear 86G meshes with the second main gear 861 of the second rotary body 86. A through-hole 864 is formed in a center of the second gear 86G and extends through the same in the front-rear direction. A first spacer 86P is disposed in the through-hole 864, and the support shaft Xf2 is inserted in the first spacer 86P. The second gear 86G is configured to rotate about the support shaft Xf2 in response to the rotation of the second rotary body 86.

The first follow gear 89 is connected to a rear end portion of the knot pusher 5. The first follow gear 89 is arranged to the right of the second gear 86G and meshes with the second gear 86G. When the first follow gear 89 rotates in response to the rotation of the second gear 86G, the knot pusher 5 connected to the first follow gear 89 also rotates. The knot pusher 5 extends forward from the first follow gear 89 and passes through the third through-hole 87C in the second rotary body support part 87.

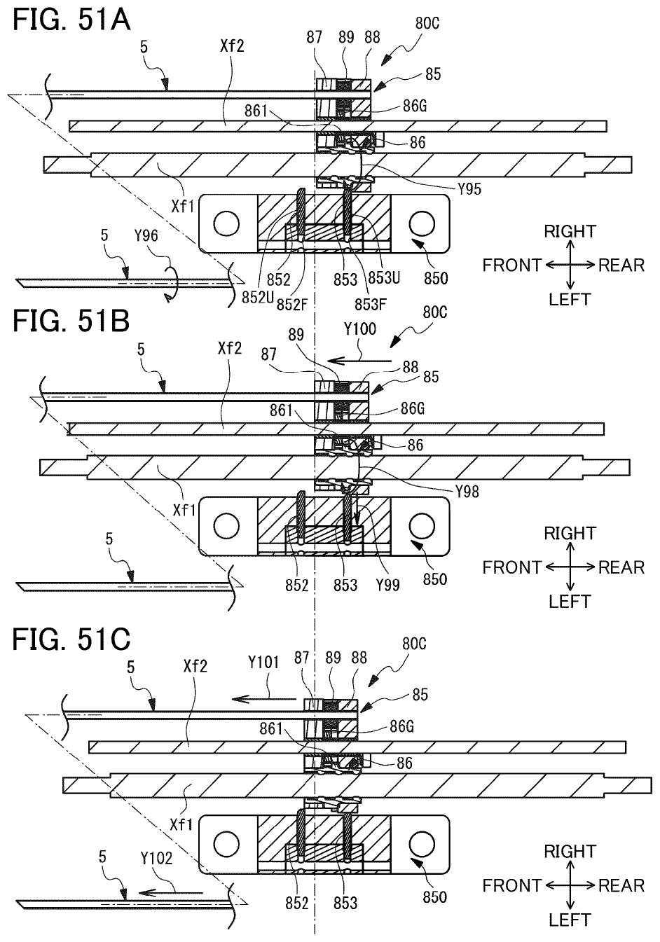

As illustrated in FIGS. 44 and 45, the second switching unit 850 is arranged to the left of the pusher support part 85 in the left-right direction. The second switching unit 850 includes a base 851, a first switching rod 852, and a second switching rod 853 that correspond to the base 801, first switching rod 802, and second switching rod 803 of the first switching unit 800 (see FIGS. 41 and 42), respectively. A right surface of the base 851 is at approximately the same position in the left-right direction as a left surface of the second rotary body support part 88. The first switching rod 852 and the second switching rod 853 are arranged further forward than the second rotary body support part 87 of the pusher support part 85. The base 851 has a first recess 852U, a second recess 853U, a first urging member 852F, and a second urging member 853F (see FIG. 51A) that correspond to the first recess 802U, second recess 803U, first urging member 802F, and second urging member 803F of the base 801 (see FIG. 49A), respectively.

### <Operations of the First Drive Mechanism 8A>

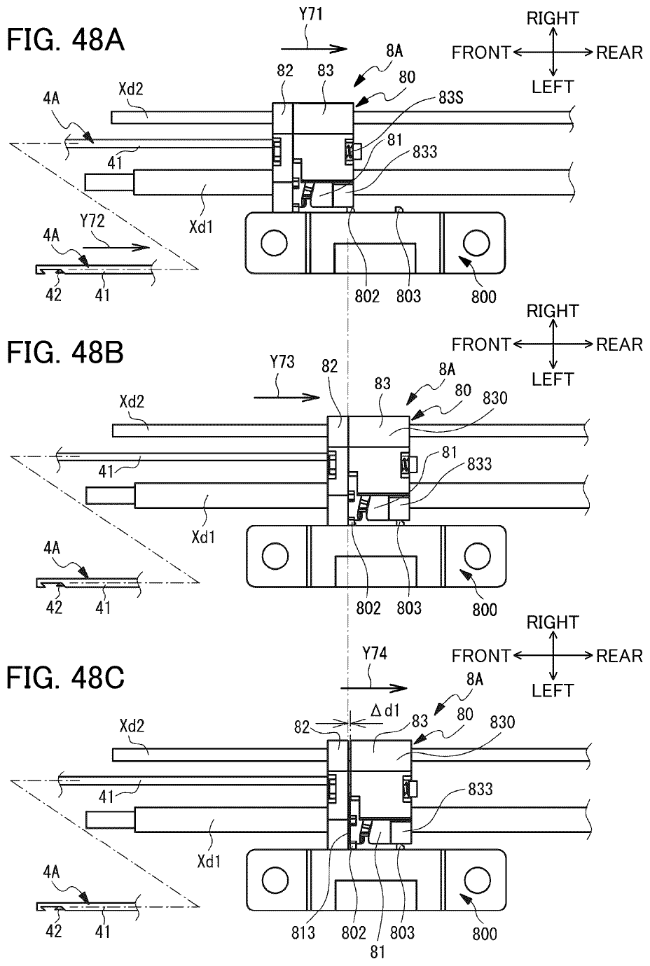

As illustrated in FIG. 48A, the first rotary body support part 82 of the first hook support part 80 is arranged farther forward than the first switching rod 802 of the first switching unit 800. The first rotary body support parts 82 and 83 of the first hook support part 80 are in close contact with each other owing to the urging forces of the first spring 82S and second spring 83S. The protrusions 813 on the first rotary body 81 illustrated in FIG. 43 are fitted in the recesses 821 in the first rotary body support part 82. Accordingly, the first rotary body 81 cannot rotate relative to the first rotary body support parts 82 and 83.

When moving the hook body 41 and wire 42 of the first retracting member 4A rearward, the ligation device 1B drives the first motor Ma1 (see FIG. 6) to rotate the first threaded shaft Xd1 in the reverse direction. Since the support shaft Xd2 is inserted in the third through-hole 82C and the first through-hole 83C, the first hook support part 80 is unable to rotate about an axis of the first threaded shaft Xd1. Therefore, the first nut portion 810 of the first rotary body 81 receives a rearward force in response to the rotation of the first threaded shaft Xd1 and moves rearward. In this case, the first rotary body support part 82 and first rotary body support part 83 supporting the first rotary body 81 move rearward (arrow Y71) in response to the movement of the first rotary body 81, as illustrated in FIG. 48A. As a result, the hook body 41 connected to the first rotary body support part 83 also moves rearward (arrow Y72).

Further, in response to the non-rotatable first rotary body 81 moving rearward, the wire 42 coupled to the first rotary body 81 via the cam follower 84A and the groove 811 also moves rearward (arrow Y72). Hence, the hook body 41 and wire 42 of the first retracting member 4A move rearward together. As the hook body 41 and wire 42 of the first retracting member 4A move rearward together, the hook 41B of the hook body 41 hooks the thread T, as illustrated in FIG. 22, for example.

As illustrated in FIG. 48B, as the first hook support part 80 moves rearward (arrow Y73) in response to the rotation of the first threaded shaft Xd1, the first switching rod 802 of the first switching unit 800 contacts the rear surface of the first rotary body support part 82 from its rear side. Thus, the first switching rod 802 restricts the rearward movement of the first rotary body support part 82. As the first threaded shaft Xd1 is further rotated in the reverse direction, only the first rotary body 81 and first rotary body support part 83 move rearward (arrow Y74), forming a gap Δd1 between the rear surface of the first rotary body support part 82 and the first rotary body 81 and between the rear surface of the first rotary body support part 82 and the front surface on the base 830 of the first rotary body support part 83, as illustrated in FIG. 48C. The protrusions 813 of the first rotary body 81 disengage from the recesses 821 in the first rotary body support part 82. The first rotary body 81 then becomes rotatable relative to the first rotary body support parts 82 and 83.

As the first threaded shaft Xd1 rotates further in the reverse direction, as illustrated in FIG. 49A, the first rotary body 81, which is now rotatable, rotates about the axis of the first threaded shaft Xd1 (arrow Y75). The first rotary body 81 rotates in the counterclockwise direction when viewed from the front side. In response to this rotation of the first rotary body 81, the cam follower 84A of the wire connector 84, which is connected to the rear end of the wire 42, moves forward along the groove 811 of the first rotary body 81, whereby the wire 42 moves forward (arrows Y76 and Y77). Since the first rotary body 81 is not moving rearward at this time, the hook body 41 connected to the first rotary body support part 83 does not move rearward. As the wire 42 moves forward relative to the hook body 41, the thread Tis gripped between the front surface 42A of the wire 42 and the hook 41B of the hook body 41, as illustrated in FIG. 22, for example.

As illustrated in FIG. 49B, as the first threaded shaft Xd1 continues to rotate in the reverse direction and the first rotary body 81 continues to rotate (arrow Y78), the second surface 812B of one of the protrusions 812 on the first rotary body 81 illustrated in FIG. 43 contacts the first switching rod 802 from below. The protrusion 812 urges the first switching rod 802 leftward. Upon receiving this force from the protrusion 812, the first switching rod 802 moves leftward against the urging force of the first urging member 802F (arrow Y79). Now, the distal end of the first switching rod 802 no longer protrudes rightward from the right surface of the base 801 in the first switching unit 800. The first rotary body support part 82 can now move rearward. The first rotary body support part 82 is moved rearward by the urging forces of the first spring 82S and the second spring 83S (arrow Y80). The first rotary body support parts 82 and 83 are now in close contact with each other, and the protrusions 813 of the first rotary body 81 are fitted into the recesses 821 of the first rotary body support part 82. The first rotary body 81 can no longer rotate relative to the first rotary body support parts 82 and 83.

As the first threaded shaft Xd1 continues to rotate in the reverse direction, the first nut portion 810 of the first rotary body 81 receives a rearward force. The first rotary body support parts 82 and 83 supporting the first rotary body 81 move rearward as the first rotary body 81 moves (arrow Y81), as illustrated in FIG. 49C. As a result, the hook body 41 connected to the first rotary body support part 83 also moves rearward (arrow Y82). Further, in response to the non-rotatable first rotary body 81 moving rearward, the wire 42 coupled to the first rotary body 81 via the cam follower 84A and the groove 811 also moves rearward (arrow Y82). Therefore, the hook body 41 and wire 42 of the first retracting member 4A move rearward together. The hook body 41 and wire 42 pass above and past the ligation target S while continuing to grip the thread T, as illustrated in FIG. 23, for example.

When the first threaded shaft Xd1 is rotated farther in the reverse direction, the second switching rod 803 of the first switching unit 800 approaches and contacts the rear surface of the first rotary body support part 82 from the rear side. The first rotary body 81 now becomes rotatable. The first rotary body 81 rotates in response to the rotation of the first threaded shaft Xd1, moving the wire 42 rearward. Since the first rotary body 81 does not move rearward at this time, the hook body 41 connected to the first rotary body support part 83 does not move rearward. As the wire 42 moves rearward relative to the hook body 41, the hook body 41 and wire 42 release the thread T from its gripped state.

As described above, the groove 811 of the first rotary body 81 and the cam follower 84A of the wire connector 84 function as a second converter 840 for converting the rotational motion of the first rotary body 81 into linear motion for moving the wire 42 forward. Further, the first switching unit 800 and the second converter 840 function as a moving mechanism 80A for moving the wire 42 relative to the hook body 41 in the front-rear direction.

### <Operations of the Third Drive Mechanism 8C>

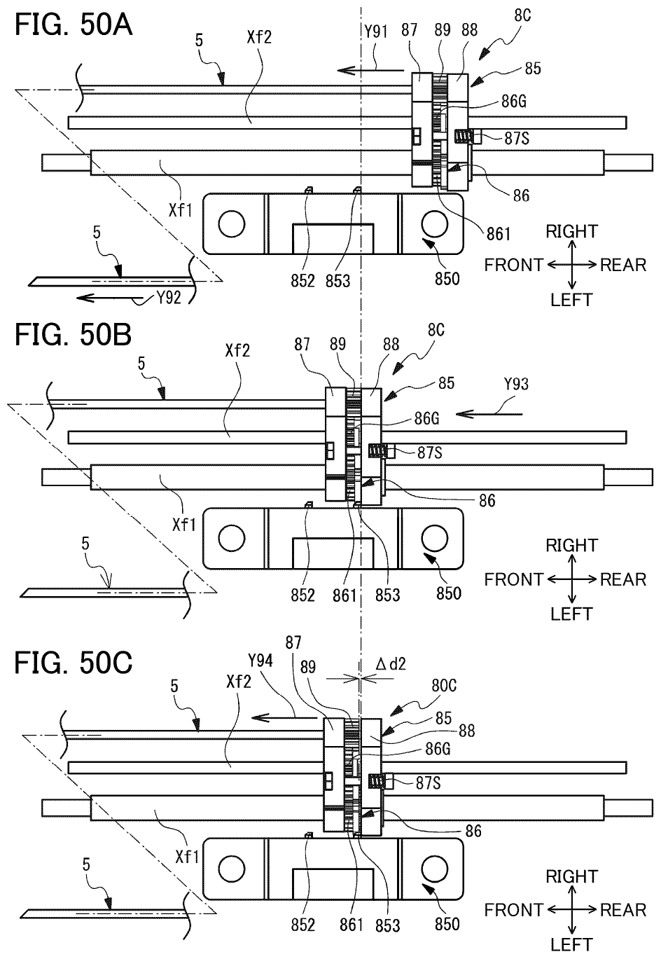

As illustrated in FIG. 50A, the second rotary body support part 87 of the pusher support part 85 is arranged further rearward than the first switching rod 852 of the second switching unit 850. The second rotary body support parts 87 and 88 of the pusher support part 85 sandwich the second rotary body 86 through the urging force of the spring 87S. The protrusions 863 on the second rotary body 86 illustrated in FIG. 46 are fitted in the recesses 881 in the second rotary body support part 88. Accordingly, the second rotary body 86 cannot move relative to the second rotary body support parts 87 and 88.

When moving the knot pusher 5 forward, the ligation device 1B drives the second motor Mc1 (see FIG. 6) to rotate the second threaded shaft Xf1 in the forward direction. Since the support shaft Xf2 is inserted in the third through-holes 87C and 88C, the pusher support part 85 is incapable of rotating about the axis of the second threaded shaft Xf1. Therefore, the second nut portion 860 of the second rotary body 86 receives a forward force in response to the rotation of the second threaded shaft Xf1 and moves forward. In this case, as illustrated in FIG. 50A, the second rotary body support parts 87 and 88 that support the second rotary body 86 move forward in response to the movement of the second rotary body 86 (arrow Y91). As a result, the knot pusher 5 connected to the first follow gear 89, which is interposed between the second rotary body support parts 87 and 88, also moves forward (arrow Y92). As the knot pusher 5 moves forward, the front end 51 of the knot pusher 5 approaches the first loop P1 formed on the first looping shaft 46 of the forming part 2B from the rear, as illustrated in FIG. 40, for example.

As illustrated in FIG. 50B, as the pusher support part 85 moves forward in response to the rotation of the second threaded shaft Xf1 (arrow Y93), the second switching rod 853 of the second switching unit 850 contacts the front surface of the second rotary body support part 88 from the front side. The second switching rod 853 thus restricts the forward movement of the second rotary body support part 88. As the second threaded shaft Xf1 rotates further in the forward direction, only the second rotary body 86 and the second rotary body support part 87 move forward (arrow Y94), as illustrated in FIG. 50C. A gap Δd2 is formed between the front surface of the second rotary body support part 88 and the second rotary body 86. The protrusions 863 on the second rotary body 86 disengage from the recesses 881 in the second rotary body support part 88. The second rotary body 86 is now rotatable relative to the second rotary body support parts 87 and 88.

As illustrated in FIG. 51A, as the second threaded shaft Xf1 continues to rotate in the forward direction, the second rotary body 86, which is now rotatable, rotates about the axis of the second threaded shaft Xf1 (arrow Y95). The second rotary body 86 rotates in the clockwise direction when viewed from the front. As the second rotary body 86 rotates, the second gear 86G, which is meshed with the second main gear 861 of the second rotary body 86, also rotates. Further, as the second gear 86G rotates, the first follow gear 89 meshed with the second gear 86G receives a force in a direction of rotation of the same. As a result, the knot pusher 5, on the rear end of which the first follow gear 89 is connected, rotates about the seventh axis C7 (arrow Y96). Since the second rotary body 86 does not move forward at this time, the knot pusher 5 supported by the second rotary body support part 87 and second rotary body support part 88 does not move forward. The knot pusher 5 rotates 180 degrees about the seventh axis C7 and passes through the thread T forming the first loop P1, as illustrated in FIG. 40, for example.

As illustrated in FIG. 51B, as the second threaded shaft Xf1 continues to rotate in the forward direction and the second rotary body 86 continues to rotate (arrow Y98), the second surface 862B of one of the protrusions 862 on the second rotary body 86 illustrated in FIG. 46 contacts the second switching rod 853 from above, whereby the protrusion 862 urges the second switching rod 853 leftward. Upon receiving a force from the protrusion 862, the second switching rod 853 moves leftward against the urging force of the second urging member 853F (arrow Y99). The distal end of the second switching rod 853 now no longer protrudes rightward from the right surface on the base 851 of the second switching unit 850. The second rotary body support part 88 can now move forward. The second rotary body support part 88 is moved forward by the urging force of the spring 87S (arrow Y100). The protrusions 863 on the second rotary body 86 are fitted into the recesses 881 in the second rotary body support part 88. The second rotary body 86 is now unable to rotate relative to the second rotary body support parts 87 and 88.

As the second threaded shaft Xf1 continues to rotate in the forward direction, the second nut portion 860 of the second rotary body 86 receives a forward force. The second rotary body support parts 87 and 88 supporting the second rotary body 86 move forward along with the movement of the second rotary body 86 (arrow Y101), as illustrated in FIG. 51C. As a result, the knot pusher 5 supported by the pusher support part 85 moves forward (arrow Y102). The knot pusher 5 moves forward after passing through the thread T forming the first loop P1, as illustrated in FIG. 40, for example.

When the second threaded shaft Xf1 is further rotated in the forward direction, the first switching rod 852 of the second switching unit 850 approaches and contacts the front surface of the second rotary body support part 88 from the front. The second rotary body 86 is now rotatable. The second rotary body 86 rotates in response to the rotation of the second threaded shaft Xf1, rotating the knot pusher 5. The knot pusher 5 is repeatedly rotated and moved forward in this way. As a result, the knot pusher 5 moves forward without catching on the first loop P1 and second loop P2, as illustrated in FIG. 40.

As described above, the second main gear 861 and second gear 86G of the second rotary body 86 and the first follow gear 89 on the knot pusher 5 function as a transmitter 890 configured to transmit the force of the second rotary body 86, which rotates in response to the rotation of the second threaded shaft Xf1, to the knot pusher 5 for rotating the same. Further, the second switching unit 850 and the transmitter 890 function as a rotating mechanism 85A configured to rotate the knot pusher 5.

### <Functions and Technical Advantages of the Ligation Device 1B>

The first drive mechanism 8A is configured to move the hook body 41 of the first retracting member 4A in the front-rear direction by rotating the first threaded shaft Xd1 while the first rotary body 81 cannot rotate relative to the first rotary body support parts 82 and 83. The first drive mechanism 8A is also configured to move the wire 42 in the front-rear direction relative to the hook body 41 by rotating the first threaded shaft Xd1 while the first rotary body 81 is rotatable relative to the first rotary body support parts 82 and 83. Thus, by driving the first motor Ma1 to rotate the first threaded shaft Xd1, the first drive mechanism 8A can move each of the hook body 41 and wire 42 of the first retracting member 4A in the front-rear direction. Accordingly, since the hook body 41 and wire 42 can both be moved in the front-rear direction with a common motor, the ligation device 1B can be made lighter and more compact.

The third drive mechanism 8C is configured to move the knot pusher 5 in the front-rear direction by rotating the second threaded shaft Xf1 while the second rotary body 86 cannot rotate relative to the second rotary body support parts 87 and 88. The third drive mechanism 8C is also configured to rotate the knot pusher 5 by rotating the second threaded shaft Xf1 while the second rotary body 86 is rotatable relative to the second rotary body support parts 87 and 88. Thus, by driving the first motor Ma1 to rotate the first threaded shaft Xd1, the third drive mechanism 8C can move the knot pusher 5 in the front-rear direction and rotate the same. Accordingly, since the ligation device 1B can both move the knot pusher 5 in the front-rear direction and rotate the knot pusher 5 with a common motor, the ligation device 1B can be made lighter and more compact.

### <Variations>

While the invention has been described in conjunction with various example structures outlined above and illustrated in the figures, various alternatives, modifications, variations, improvements, and/or substantial equivalents, whether known or that may be presently unforeseen, may become apparent to those having at least ordinary skill in the art. Accordingly, the example embodiments of the disclosure, as set forth above, are intended to be illustrative of the invention, and not limiting the invention. Various changes may be made without departing from the spirit and scope of the disclosure. Therefore, the disclosure is intended to embrace all known or later developed alternatives, modifications, variations, improvements, and/or substantial equivalents. Some specific examples of potential alternatives, modifications, or variations in the described invention are disposed below:

The holder 3 in the ligation devices 1A and 1B is not limited to the described configuration having the first jaw part 3A and the second jaw part 3B. The holder 3 may have another configuration capable of holding the ligation target S. For example, the holder 3 may hold the ligation target S by adsorbing the ligation target S to the distal end of the body 2A or by wrapping around the ligation target S. The first retracting member 4A of the ligation devices 1A and 1B is not limited to the described configuration having the hook body 41 and the wire 42. The first retracting member 4A may have another configuration capable of drawing thread T into the body 2A. For example, the first retracting member 4A may possess only the hook body 41 and not the wire 42. The first retracting member 4A may draw the thread T into the body 2A by gripping and moving the thread T rearward, for example. The same applies to the second retracting member 4B.

The ligating process in the above-described embodiments is merely an example, and the ligation target S may be ligated with the thread T according to another method. The repositioning process in the above-described embodiments is merely an example, and the thread T may be repositioned according to another method. For example, the operator may reposition the thread T manually.

The forming part 2B for forming the first loop P1 and second loop P2 in the ligation devices 1A and 1B is not limited to the configuration in the above embodiments. For example, the ligation devices 1A and 1B need not possess a forming part 2B, and loops may be formed in the thread T through operations of the retractor 4 in the body 2A.

The ligation devices 1A and 1B may possess just the first retracting member 4A and not the second retracting member 4B. The ligation devices 1A and 1B may also possess one or more other retracting members, in addition to the first retracting member 4A and second retracting member 4B. In other words, the ligation devices 1A and 1B may possess three or more retracting members. The ligation devices 1A and 1B may simply possess the retractor 4 and a drive mechanism for the retractor 4, and need not possess the knot pusher 5 and the third drive mechanisms 7C and 8C. The threaded shafts X1 may also be slide screws or ball screws.

The first drive mechanism 7A and the second drive mechanism 7B of the ligation device 1A move the retractor 4 in the front-rear direction by rotating the threaded shafts X1 to move the first hook support part 70A and the second hook support part 70B. Alternatively, the first drive mechanism 7A and second drive mechanism 7B may move the retractor 4 in the front-rear direction by moving the first hook support part 70A and second hook support part 70B without use of the threaded shafts X1. For example, the first drive mechanism 7A and second drive mechanism 7B may have a timing belt supported at both ends in the front-rear direction by two pulleys. The first hook support part 70A and second hook support part 70B may be disposed on the timing belt. The ligation device 1A may move the retractor 4 in the front-rear direction by circulating the timing belt to move the first hook support part 70A and second hook support part 70B. Still alternatively, the first drive mechanism 7A and second drive mechanism 7B may move the retractor 4 in the front-rear direction by moving the first hook support part 70A and second hook support part 70B with a linear motor, for example. The same applies to the third drive mechanism 7C. The same is also applicable to the first drive mechanism 8A, second drive mechanism 8A′, and third drive mechanism 8C of the ligation device 1B.

The first drive mechanism 7A need not possess the first follow gear 71C. The first main gear 71B may be meshed with the follow gear 719. Pulleys may be used in place of the first main gear 71B and first follow gear 71C, and a belt may be stretched around the pulleys. The wire 42 may then be moved in the front-rear direction as the belt circulates in response to the rotation of the first auxiliary shaft Xa2. The same applies to the second drive mechanism 7B, first drive mechanism 8A, and second drive mechanism 8A′.

A gear coupled to the rotational shaft of the first auxiliary motor Ma2 may be used in place of the first auxiliary shaft Xa2 and the first main gear 71B of the first drive mechanism 7A. The gear may extend in the front-rear direction and be meshed with the first follow gear 71C. In this case, the first drive mechanism 7A need not possess the first auxiliary shaft Xa2 and the first main gear 71B. The same applies to the second drive mechanism 7B, first drive mechanism 8A, and second drive mechanism 8A′.

Pulleys may be employed in the third drive mechanism 7C in place of the second main gear 73B and the second follow gear 739. A belt may be stretched around the pulleys. The knot pusher 5 may be rotated by rotating the second auxiliary shaft Xc2 to circulate the belt. Alternatively, a gear coupled to the rotational shaft of the second auxiliary motor Mc2 may be used in place of the second auxiliary shaft Xc2 and the second main gear 73B of the third drive mechanism 7C. The gear may extend in the front-rear direction and be meshed with the second follow gear 739. In this case, the third drive mechanism 7C need not possess the second auxiliary shaft Xc2 and the second main gear 73B.

The first drive mechanism 8A may have a gear disposed on the side surface of the first rotary body 81 in the first hook support part 80. As the wire connector 84 that connects to the wire 42, the first hook support part 80 may have a worm gear meshed with the gear on the side surface of the first rotary body 81. The first hook support part 80 may move the wire 42 in the front-rear direction by having the gear on the side surface of the first rotary body 81 move the worm gear in the front-rear direction when the first rotary body 81 is rotated in response to the rotation of the first threaded shaft Xd1. Further, in the ligation device 1B, the first switching unit 800 switches the first rotary body 81 between a rotatable state and a non-rotatable state in response to the first hook support part 80 moving in the front-rear direction. The first rotary body 81 may be configured to switch between the rotatable state and the non-rotatable state according to another method. For example, an actuator capable of forcibly separating the first rotary body support parts 82 and 83 may be used to switch the first rotary body 81 between the rotatable state and the non-rotatable state at suitable timings. Alternatively, the first drive mechanism 8A may possess just the first threaded shaft Xd1 and not the support shaft Xd2. The hook body 41 may be connected to the first rotary body support part 82 of the first hook support part 80.

The third drive mechanism 8C may include a pulley in place of the first follow gear 89 of the pusher support part 85. A belt may be stretched around the second rotary body 86 and the pulley. The knot pusher 5 connected to the pulley may be configured to rotate as the second rotary body 86 rotates to circulate the belt. Further, in the ligation device 1B, the second switching unit 850 switches the second rotary body 86 between a rotatable state and a non-rotatable state as the pusher support part 85 moves in the front-rear direction. The second rotary body 86 may be switched between the rotatable state and the non-rotatable state according to another method. For example, an actuator capable of forcibly separating the second rotary body support parts 87 and 88 may be configured to switch the second rotary body 86 between the rotatable state and the non-rotatable state at suitable timings. Alternatively, the third drive mechanism 8C may possess just the second threaded shaft Xf1 and not the support shaft Xf2.

Portions of the first hook support part 70A and the second hook support part 70B may be arranged at the same position in directions orthogonal to the front-rear direction (i.e., in the up-down and left-right directions). When viewed from the rear, the portions of the first hook support part 70A and second hook support part 70B may overlap each other.

The first hook support part 70A may always be arranged farther forward than the pusher support part 70C. In this case, the distance L13 may be longer than the distance L11. The first hook support part 70A and the pusher support part 70C may be at the same position in the front-rear direction. In this case, the distances L11 and L13 may be equal to each other.

The robot connector 9 may be arranged to the rear of the drive motors Mm and to the front of the case 704 or may be arranged on the rear end of the case 704. The robot R need not drive all of the second jaw part 3B, feeder 6, and forming part 2B with the robot motors MR, but may operate one or more of the second jaw part 3B, feeder 6, and forming part 2B with the robot motors MR. The ligation devices 1A and 1B do not need to be connected to the robot R to be used, but may be capable of being operated independently. In this case, the control wires for operating the second jaw part 3B, feeder 6, and forming part 2B may be directly operated by an operator or by motors built into the ligation devices 1A and 1B. For example, the ligation devices 1A and 1B may be used with the rear end of the case 704 directly supported by the operator.

The front-rear direction is an example of “longitudinal direction” of the disclosure. The body 2A is an example of “body” of the disclosure. The holder 3 is an example of “holder” of the disclosure. The retractor 4 is an example of “retractor” of the disclosure. The drive units 7 and 8 are examples of “drive unit” of the disclosure. The forming part 2B (first loop shaft 46, second loop shaft 56) is an example of an example of “looping shaft” of the disclosure. The knot pusher 5 is an example of “knot pusher” of the disclosure. The robot connector 9 is an example of “robot connector” of the disclosure. The feeder 6 is an example of “feeder” of the disclosure.

What is claimed is:

1. A ligation device comprising:
a body having a cylindrical shape and extending in a longitudinal direction, the body having one end and another end opposite each other in the longitudinal direction;
a holder disposed at the one end of the body and configured to hold a ligation target;
a retractor having a part accommodated inside the body, the retractor being movable toward the other end of the body in the longitudinal direction to draw a thread into an interior of the body, the thread being to be used for ligating the ligation target held by the holder; and
a drive unit connected to the other end of the body, the drive unit including a first motor for moving the retractor in the longitudinal direction relative to the body.

2. The ligation device according to claim 1,
wherein the retractor includes a first retractor, the first retractor including a hook body having a distal end and a base end opposite each other in the longitudinal direction, the distal end being formed with a first hook for hooking the thread, and
wherein the drive unit includes a first drive mechanism configured to move the first retractor in the longitudinal direction relative to the body, the first drive mechanism including:
the first motor;
a first threaded shaft configured to rotate when the first motor is driven; and
a first hook support part supporting the base end of the hook body of the first retractor, the first hook support part having a first nut portion meshed with the first threaded shaft, the first nut portion being movable in the longitudinal direction in response to rotation of the first threaded shaft to move the first hook support part in the longitudinal direction.

3. The ligation device according to claim 2,
wherein the first retractor further includes a wire configured to hold the thread hooked by the first hook of the hook body, and
wherein the first drive mechanism is further configured to move the wire in the longitudinal direction relative to the hook body.

4. The ligation device according to claim 3,
wherein the first drive mechanism further includes:
an auxiliary motor;
an auxiliary shaft extending parallel to the first threaded shaft and configured to rotate when the auxiliary motor is driven;
a main gear rotatable in response to rotation of the auxiliary shaft;
a follow gear meshed with the main gear and rotatable in response to rotation of the main gear; and
a wire connector connected to a base end of the wire in the longitudinal direction, the wire connector being meshed with the follow gear and being configured to convert rotation of the follow gear into linear motion to move the wire in the longitudinal direction relative to the hook body.

5. The ligation device according to claim 3,
wherein the first hook support part includes:
a rotary body having the first nut portion;
a wire connector connected to a base end of the wire in the longitudinal direction; and
a rotary body support part supporting the rotary body and connected to the base end of the hook body, the rotary body being switchable between a rotatable state and a non-rotatable state relative to the rotary body support part,
wherein, while the rotary body is in the non-rotatable state relative to the rotary body support part,
the rotary body moves in the longitudinal direction in response to rotation of the threaded shaft to move both the rotary body support part and the wire connector in the longitudinal direction, and
wherein, while the rotary body is in the rotatable state relative to the rotary body support part,
the rotary body rotates in response to rotation of the first threaded shaft, and
rotation of the rotary body is converted into linear motion to move the wire in the longitudinal direction relative to the hook body.

6. The ligation device according to claim 2,
wherein the retractor further includes a second retractor, the second retractor including a hook body having a distal end and a base end opposite each other in the longitudinal direction, the distal end in the longitudinal direction of the second retractor being formed with a second hook for hooking the thread,
wherein the drive unit further includes a second drive mechanism configured to move the second retractor in the longitudinal direction relative to the body, the second drive mechanism including:
another first motor;
another first threaded shaft configured to rotate when the another first motor is driven; and
a second hook support part supporting the base end of the hook body of the second retractor, the second hook support part having another first nut portion meshed with the another first threaded shaft, the another first nut portion being movable in the longitudinal direction in response to rotation of the another first threaded shaft to move the second hook support part in the longitudinal direction, and
wherein the first hook support part and the second hook support part are at different positions from each other in a direction orthogonal to the longitudinal direction.

7. The ligation device according to claim 2, further comprising:
a looping shaft configured to rotate to form a loop in the thread drawn into the interior of the body by the first retractor; and
a knot pusher having a tubular shape extending in the longitudinal direction, the first retractor being accommodated inside the knot pusher, the knot pusher being movable in the longitudinal direction toward the one end of the body to push the loop formed by the looping shaft toward the one end of the body,
wherein the drive unit further includes a knot-pusher drive mechanism configured to move the knot pusher in the longitudinal direction relative to the body, the knot-pusher drive mechanism including:
a second motor;
a second threaded shaft configured to rotate when the second motor is driven; and
a pusher support part supporting a base end of the knot pusher in the longitudinal direction, the pusher support part having a second nut portion meshed with the second threaded shaft, the second nut portion being movable in the longitudinal direction in response to rotation of the second threaded shaft to move the pusher support part in the longitudinal direction.

8. The ligation device according to claim 7,
wherein the knot-pusher drive mechanism further includes:
an auxiliary motor;
an auxiliary shaft extending parallel to the second threaded shaft, the auxiliary shaft being configured to rotate when the auxiliary motor is driven;
a main gear configured to rotate in response to rotation of the auxiliary shaft; and
a follow gear meshed with the main gear and configured to rotate in response to rotation of the main gear, the follow gear being connected to the base end of the knot pusher, rotation of the follow gear causing the knot pusher to rotate about an axis passing through a center of the knot pusher and extending in the longitudinal direction.

9. The ligation device according to claim 7,
wherein the pusher support part includes:
a rotary body having the second nut portion;
a pusher connector coupled to the rotary body and connected to the base end of the knot pusher; and
a rotary body support part supporting the rotary body, the rotary body being switchable between a rotatable state and a non-rotatable state relative to the rotary body support part,
wherein, while the rotary body is in the non-rotatable state relative to the rotary body support part,
the rotary body moves in the longitudinal direction in response to rotation of the second threaded shaft to move the pusher connector in the longitudinal direction, and
wherein, while the rotary body is in the rotatable state relative to the rotary body support part,
the rotary body rotates in response to rotation of the second threaded shaft to cause the knot pusher to rotate about an axis passing through a center of the knot pusher and extending in the longitudinal direction.

10. The ligation device according to claim 7,
wherein the first hook support part has a part to which the base end of the hook body of the first retractor is connected,
wherein the pusher support part has a part to which the base end of the knot pusher is connected, and
wherein the part of the first hook support part and the part of the pusher support part overlap each other when viewed in the longitudinal direction.

11. The ligation device according to claim 7,
wherein a distance in the longitudinal direction between the body and the pusher support part is shorter than a distance in the longitudinal direction between the body and the first hook support part.

12. The ligation device according to claim 2, further comprising a robot connector connectable to a robot,
wherein a distance in the longitudinal direction between the robot connector and the first motor is shorter than each of:
a distance in the longitudinal direction between the robot connector and the first threaded shaft; and
a distance in the longitudinal direction between the robot connector and the first hook support part.

13. The ligation device according to claim 1,
wherein the holder includes:
a first jaw part; and
a second jaw part movable between an adjacent position and a separated position relative to the first jaw part, the second jaw part being positioned adjacent to the first jaw part and holding the ligation target while being in the adjacent position, the second jaw part being separated from the first jaw part while being in the separated position,
the ligation device further comprising:
a feeder disposed in the first jaw part and configured to feed the thread toward the second jaw part;
a looping shaft configured to rotate to form a loop in the thread when the thread fed by the feeder is drawn into the body by the first retractor; and
a robot connector connectable to a robot incorporating a robot motor, at least one of the second jaw part, the feeder and the looping shaft being configured to be driven by the robot motor.
